(19) United States
(12) Patent Application Publication
(10) Pub. No.: US 2025/0000888 A1
(43) Pub. Date: Jan. 2, 2025

(54) METHODS OF TREATING MYELODYSPLASTIC SYNDROMES WITH DECITABINE AND CEDAZURIDINE

(71) Applicant: OTSUKA PHARMACEUTICAL CO., LTD., Tokyo (JP)

(72) Inventors: Harold Keer, Pleasanton, CA (US); Mohammad Azab, San Francisco, CA (US); Yong Hao, Vancouver (CA)

(21) Appl. No.: 18/693,727

(22) PCT Filed: Sep. 21, 2022
(86) PCT No.: PCT/US2022/076757

§ 371 Date: Mar. 20, 2024

Related U.S. Application Data
(60) Provisional application No. 63/246,547, filed on Sep. 21, 2021.

(51) Int. Cl. A61K 31/7068; A61K 9/00; A61K 9/20; A61K 9/28; A61P 35/02

(52) U.S. Cl. CPC A61K 31/7068; A61K 9/0053; A61K 9/2009; A61K 9/2018; A61K 9/2054; A61K 9/28; A61P 35/02

(57) ABSTRACT

Provided herein are methods of treating myelodysplastic syndromes (MDS) in a subject in need thereof. Also provided herein are methods of treating lower-risk MDS and chronic myelomonocytic leukemia (CMML) in a subject in need thereof. Also provided herein are methods of treating lower-risk MDS and chronic myelomonocytic leukemia (CMML) in a subject having a TP53 mutation in need thereof. Such methods include administering to the subject an effective amount of cedazuridine and an effective amount of decitabine, thereby treating the myelodysplastic syndrome. Such methods may increase the subject's survival (e.g., overall median survival) to about 130% to about 400% relative to survival obtained by treatment with a hypomethylating agent alone.

### STATEMENT OF PRIORITY

This application claims the benefit of U.S. Provisional Application Ser. No. 63/246,547, filed Sep. 21, 2021, the entire contents of which are incorporated by reference herein.

### FIELD OF THE INVENTION

The present invention relates to methods for the treatment of cancer. In particular, the present invention relates to methods for the treatment of myelodysplastic syndromes.

### BACKGROUND OF THE INVENTION

Myelodysplastic syndromes (MDS) are a group of bone marrow disorders characterized by a lack of healthy blood cells. Patients with MDS typically have low blood cell counts that may cause infection, anemia, spontaneous bleeding, and/or bruising. In addition, in patients with MDS, mature blood cells may be dysplastic and so may not function properly. In order to determine prognosis and treatment regimens for the various MDS stages, scoring systems have been developed. In the original International Prognostic Scoring System (IPSS), a patient's score is determined based on the percentage of leukemic blast cells in the patient's marrow, the type of chromosomal changes, if any, in the patient's marrow cells, and the presence of one or more cytopenias. In the revised International Prognostic Scoring System (IPSS-R), a patient's score is determined based on the percentage of blasts in the patient's bone marrow, the type and number of chromosome abnormalities in the patient's cells, and the level of red blood cells, platelets, and/or neutrophils in the patient's blood. In the World Health Organization (WHO) Prognostic Scoring System (WPSS), a patient's score is determined based on the type of MDS the patient has (based on the WHO classification system), the type of chromosomal abnormalities, and whether or not the patient needs regular blood transfusions.

Decitabine (5-aza-2′-deoxycytidine), a cytidine analog, is an antineoplastic agent and hypomethylating agent (HMA) that has been used for the treatment of MDS.

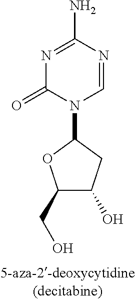

Cedazuridine ((4R)-2′-deoxy-2′,2′-difluoro-3,4,5,6-tetrahydrouridine; also known as E7727) is a cytidine deaminase (CDA) inhibitor. Cedazuridine and methods of making and/or using thereof are further disclosed in U.S. Pat. Nos. 8,268,800 and 9,834,576, the contents of which are incorporated by reference herein in their entirety.

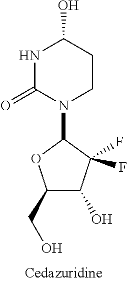

Astex Pharmaceuticals, Inc. received FDA approval for a fixed dose combination of decitabine and cedazuridine, which is sold under the brand name INQOVI© by Taiho Oncology, Inc., for the treatment of adults with MDS.

MDS has also been treated by the administration of hypomethating agents alone. In one randomized clinical study, patients were either treated with decitabine (15 mg/m² given intraveneously over 3 hours, every 8 hours for 3 days) or given best supportive care (BSC) measures only. The intention-to-treat analysis indicated that median survival was not significantly different between patients treated with decitabine and those who only received supportive care measures (14.0 months vs. 14.9 months). See, Kantarjian H, et al., Decitabine improves patient outcomes in myelodysplastic syndromes, Cancer, Apr. 15, 2006, 106 (8). In another clinical trial by the European Organisation for Research and Treatment of Cancer (EORTC), intermediate or high-risk MDS patients age 60 or older were either treated with low-dose decitabine (administered intravenously over 4 hours, 3 times per day for three days per 6 week cycle) or received BSC measures only. Prolongation of overall survival (OS) with decitabine was not determined to be statistically significant over treatment with BSC (OS=10.1 months vs. 8.5 months, respectively). See, Lübbert M, et al., Low-dose decitabine versus best supportive care in elderly patients with intermediate- or high-risk myelodysplastic syndrome (MDS) ineligible for intensive chemotherapy: final results of the randomized phase III study of the European Organisation for Research and Treatment of Cancer Leukemia Group and the German MDS Study Group, J Clin Oncol, 2011 May 20; 29 (15): 1987-96.

Azacitidine (or 5-azacytidine) is another hypomethylating agent that has been used to treat MDS. In one randomized, multicenter clinical trial (CALB 9221), patients with MDS were randomized between azacitidine and best supportive care (BSC) measures only. The median time to leukemic transformation or death was 21 months in patients treated with azacitidine versus 12 months in the BSC arm. See, Silverman L R, et al., Randomized controlled trial of azacitidine in patients with the Myelodysplastic Syndrome: A study of the Cancer and Leukemia Group B, J Clin Oncol 2002:20(10):2429-2440. A randomized study with CC-486, an oral formulation of azacitidine, in the IPPS Int-1 population showed a median overall survival of approximately 17 months for both placebo and treated patients. See, Garcia-Manero, et al, Phase III, Randomized, Placebo-Controlled Trial of CC-486 (Oral Azacitidine) in Patients with Lower-Risk Myelodysplastic Syndromes, J. Clin. Onc. (2021) 39:13, 1426-1436.

Lower-risk (IPSS Low Risk and Int-1 Subgroups) MDS patients are typically treated supportively to address cytopenias. DNA methyltransferase inhibitors such as azacitidine and decitabine are FDA-approved for higher risk MDS patients, and while the decitabine labeling includes IPSS Int-1 patients, it is not widely used in this population.

Chronic Myelomonocytic Leukemia (CMML) is an uncommon MDS/MPN overlap syndrome that has historically been included under the umbrella of myelodysplastic syndromes (MDS) for clinical trial and treatment. As a result, DNA methyltransferase inhibitors (DNMTi) such as decitabine or azacitidine have been the established standard of care for the treatment of CMML.

Approved intravenous or subcutaneous regimens require 5-7 days of treatment every month, burdening older cancer patients due to daily travel and treatment time and may increase potential risk from pandemic SARS-CoV-2 infection. In the context of pandemic SARS-CoV-2, parenteral therapy also increases contact with medical settings with increased infection risk. Because DNA methyltransferase inhibitors are rapidly degraded by cytidine deaminase in the gut and liver, oral availability has only been recently possible.

### SUMMARY OF THE INVENTION

Provided according to embodiments of the invention are methods of treating myelodysplastic syndromes (MDS), including higher-risk MDS or lower-risk MDS, in a subject in need thereof. In some embodiments, such methods include administering to the subject an effective amount of cedazuridine and an effective amount of decitabine, thereby treating the MDS. In some embodiments, administering an effective amount of cedazuridine and an effective amount of decitabine increases the subject's survival (e.g., overall median survival) to about 130% to about 400% relative to survival obtained by treatment with a hypomethylating agent alone. In some embodiments, the MDS is lower-risk MDS. In some embodiments, the MDS is chronic myelomonocytic leukemia (CMML).

In some embodiments, administering an effective amount of cedazuridine and an effective amount of decitabine increases the MDS subject's survival to about 200% to about 400%, relative to survival obtained when the subject only receives best supportive care (BSC).

In some embodiments, administering an effective amount of cedazuridine and an effective amount of decitabine to a subject with MDS provides the subject with a survival of at least about 28, 30, 32, 34, or 36 months. In some embodiments, administering an effective amount of cedazuridine and an effective amount of decitabine to a subject with MDS provides the subject with a leukemia-free survival of at least about 26, 28, 30, 32, or 34 months.

In some method embodiments of the invention, treatment of lower-risk MDS includes treatment of a lower-risk MDS subject as determined by the International Prognostic Scoring System (IPPS), including the Low and Intermediate-1 subgroups. In some method embodiments of the invention, treatment of lower-risk MDS includes treatment of a lower-risk MDS subject as determined by the International Prognostic Scoring System-Revised (IPPS-R), including the Very Low, Low, and Intermediate subgroups. In some method embodiments of the invention, treatment of lower-risk MDS includes treatment of a lower-risk MDS subject as determined by the WHO Classification-based Prognostic Scoring System (WPSS), including the Very Low, Low, and Intermediate subgroups. In some method embodiments of the invention, treatment of lower-risk MDS includes treatment of a lower-risk MDS subject as determined by the French-American-British (FAB) criteria.

In some embodiments of the invention, administering an effective amount of cedazuridine and an effective amount of decitabine to a subject includes administering to a subject cedazuridine and decitabine in one or more oral dosage forms. In some embodiments, administering an effective amount of cedazuridine and an effective amount of decitabine to a subject includes administering a single solid oral dosage form that includes cedazuridine and decitabine.

### BRIEF DESCRIPTION OF THE DRAWINGS

FIG. 1 shows patient randomization and disposition. CMML, chronic myelomonocytic leukemia; MDS, myelodysplastic syndrome.

FIGS. 2A-2B show mean plasma decitabine concentration-time profiles following single and multiple doses of IV decitabine and Oral decitabine/cedazuridine on Days 1, 2, and 5.

Linear and semi-log scales. A: Mean plasma decitabine concentration-time profiles on days 1, 2, and 5. B: Lower limit of quantification (LLOQ) of plasma decitabine concentration-time profiles on days 1, 2, and 5.

FIG. 3 is a Kaplan-Meier plot for leukemia-free survival of the subjects in a Phase 3 trial for the use of oral decitabine/cedazuridine to treat MDS.

FIG. 4 is a Kaplan-Meier plot for the overall survival of the subjects in a Phase 3 trial for the use of oral decitabine/cedazuridine to treat MDS.

FIG. 5 is a Kaplan-Meier plot for leukemia-free survival of the subjects with CMML or Int-1 or low risk MDS in a Phase 3 trial for the use of oral decitabine/cedazuridine to treat MDS.

FIG. 6 is a Kaplan-Meier plot for the overall survival of the subjects with CMML or Int-1 or low risk MDS in a Phase 3 trial for the use of oral decitabine/cedazuridine to treat MDS.

FIG. 7 shows a Kaplan-Meier plot for the TP53 mutation population compared to the wild-type population.

FIG. 8 shows a Kaplan-Meier plot for the mono-allelic TP53 mutation population compared to the bi-allelic TP53 population.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations, and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention.

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, 5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

“Effective amount” refers to the amount required to produce a desired effect (e.g., enhancing the half-life, bioavailability or efficacy of a therapeutic agent treating cancer in a subject, or reducing DNA methylation in a subject).

“Pharmaceutically acceptable” refers to those properties and/or substances that are acceptable to the patient from a pharmacological and/or toxicological point of view, and/or to the manufacturing pharmaceutical chemist from a physical and/or chemical point of view regarding composition, formulation, stability, patient acceptance, bioavailability, and compatibility with other ingredients.

“Pharmaceutically acceptable salt” refers to an acid or base salt of a compound of the invention, which salt possesses the desired pharmacological activity and is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include without limitation ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quaternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides.

“Pharmaceutically acceptable excipient” can mean any substance, not itself a therapeutic agent, used as a carrier, diluent, adjuvant, binder, and/or vehicle for delivery of a therapeutic agent to a subject, or added to a pharmaceutical composition to improve its handling or storage properties or to permit or facilitate formation of a compound or composition into a unit dosage form for administration.

“Unit dosage form” refers to a physically discrete unit suitable as a unitary dosage for human or other animal subjects. Each unit dosage form may contain a predetermined amount of an active substance (e.g., cedazuridine and/or decitabine) calculated to produce a desired effect.

“Optional” or “optionally” means that the subsequently described event or circumstance may or may not occur, and that the description includes instances where the event or circumstance occurs and instances in which it does not. For example, an alkyl that is “optionally substituted” encompasses both an alkyl that is unsubstituted and an alkyl that is substituted.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, fifteen-fold, etc.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

“Subject,” as used herein, refers to a human. A subject may also be referred to as a patient.

By the term “treat,” “treating,” or “treatment of” (or grammatically equivalent terms), it is meant that the severity of the subject's condition is reduced or at least partially improved or ameliorated and/or that some alleviation, mitigation or decrease in at least one clinical symptom is achieved. “Treating” in reference to a disease, disorder or condition may refer to: (i) inhibiting a disease, disorder or condition, e.g., arresting its development, (ii) relieving a disease, disorder or condition, e.g., causing regression of the clinical symptoms; and/or (iii) stabilizing or controlling the progression of a disease, disorder or condition, including delaying progression to AML, or preventing a relapse or disease progression after a reduction in or absence of a detectable level of disease. A subject is “in need of” treatment for MDS, higher-risk MDS, or lower-risk MDS if the subject has MDS, higher-risk MDS, or lower-risk MDS, respectively, as determined by the IPSS, IPSS-R, WPSS, and/or FAB. A subject is “in need of” treatment for chronic myelomonocytic leukemia (CMML) if the subject meets the French-American-British (FAB) classification for CMML, or any other known criteria for identifying CMML.

The term “administering” or “administration” of a compound and/or composition of the present invention to a subject includes a route of introducing or delivering to a subject a compound to perform its intended function.

As used herein, “lower-risk MDS” refers to MDS in subjects that are deemed lower-risk by the International Prognostic Scoring System (IPSS), International Prognostic Scoring System-Revised (IPSS-R), and/or the WHO Classification-based Prognostic Scoring System (WPSS).

As such, in some embodiments, a lower-risk MDS subject may be classified as Low or Intermediate-1 (also referred to herein as Int-1) as determined by the IPSS; Very Low, Low, or Intermediate, as determined by the IPSS-R; and/or Very Low, Low, or Intermediate, as determined by the WPSS. Lower-Risk MDS may also be determined using the French-American-British (FAB) criteria.

A “cycle,” as used herein, refers to 28 consecutive days. In some embodiments, cedazuridine and decitabine are administered for days 1-5 of each 28-day cycle. Multiple cycles or a plurality of cycles may be performed, either consecutively or with a break between cycles. Additionally, other cycle lengths and different dosing regimens may also be used.

A “hypomethylating agent,” as used herein (also known as DNA methyltransferase or DNMT inhibitors), refers to an administered drug that inhibits or reduces DNA methylation.

Examples of hypomethylating agents include, but are not limited to, decitabine, cytidine, azacitidine (5-azacytidine), and guadecitabine.

As used herein, a subject's “overall survival time” is the number of days (or months) from the date of randomization in a clinical trial, or the first day of the subject's treatment with decitabine and cedazuridine, to the date of death for the subject.

As used herein, the “median overall survival time” is the number of days (or months) from the date of randomization in a clinical trial, or the first day of the subject's treatment with decitabine and cedazuridine, or the first day of the subject's treatment, until half of the patients remain alive, as determined by Kaplan-Meier estimates.

As used herein, a subject's “leukemia-free survival time” is the number of days (or months) from the date of randomization to the date when bone marrow or peripheral blood blasts reach ≥20%, or death.

As used herein, a subject's “median leukemia-free survival time” is the number of days (or months) from the date of randomization to the date when half of the patients have bone marrow or peripheral blood blasts ≥20%, or have died, as determined by Kaplan-Meier estimates.

As used herein, “best supportive care” or “BSC” refers to any care or treatment as needed for the subject that improves or optimizes the subject's quality of life but is not a chemotherapeutic agent (e.g., a hypomethylating agent) for the treatment of MDS. Examples of BSC include the administration of narcotic or non-narcotic analgesics, corticosteroids, and gastrointestinal medications.

### Methods of Treating MDS

Provided according to embodiments of the invention are methods of treating myelodysplastic syndromes (MDS), including higher-risk or lower-risk MDS, in a subject in need thereof. In some embodiments, such methods include administering to the subject an effective amount of cedazuridine and an effective amount of decitabine, thereby treating the MDS. In some embodiments, administering an effective amount of cedazuridine and an effective amount of decitabine increases the subject's survival (e.g., overall median survival) to about 130% to about 400% relative to survival obtained by treatment with a hypomethylating agent alone. As such, in some embodiments, administering an effective amount of cedazuridine and an effective amount of decitabine increases the subject's survival to about 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%3, 325%, 330%, 335%, 340%, 345%, 350%, 355%, 360%, 365%, 370%, 375%, 380%, 385%, 390%, 395%, or 400% or more, or any range defined therebetween, relative to survival obtained by treatment with a hypomethylating agent alone. In some embodiments, administering an effective amount of cedazuridine and an effective amount of decitabine increases the subject's survival (e.g., overall median survival) at least about 130%, at least about 200%, at least about 250%, at least about 300%, at least about 320%, and/or at least about 330%, relative to survival obtained by treatment with a hypomethylating agent alone.

In some embodiments, administering an effective amount of cedazuridine and an effective amount of decitabine increases the subject's leukemia-free survival (e.g., median leukemia-free survival) to about 130% to about 400% relative to leukemia-free survival obtained by treatment with a hypomethylating agent alone. As such, in some embodiments, administering an effective amount of cedazuridine and an effective amount of decitabine increases the subject's leukemia-free survival to about 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400% or more, or any range defined therebetween, relative to leukemia-free survival obtained by treatment with a hypomethylating agent alone. In some embodiments, administering an effective amount of cedazuridine and an effective amount of decitabine increases the subject's leukemia-free survival (e.g., median leukemia-free survival) at least about 130%, at least about 200%, at least about 250%, at least about 300%, at least about 320%, and/or at least about 330%, relative to the leukemia-free survival obtained by treatment with a hypomethylating agent alone.

In some embodiments, administering an effective amount of cedazuridine and an effective amount of decitabine increases the MDS subject's survival (e.g., overall median survival) to about 200% to about 400% relative to survival obtained when the subject only receives best supportive care (BSC). As such, in some embodiments, administering an effective amount of cedazuridine and an effective amount of decitabine increases the subject's survival to about 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400%, relative to survival obtained with BSC measures alone.

In some embodiments, administering an effective amount of cedazuridine and an effective amount of decitabine increases the MDS subject's leukemia-free survival (e.g., median leukemia-free survival) to about 200% to about 400% relative to the leukemia-free survival obtained when the subject only receives best supportive care (BSC). As such, in some embodiments, administering an effective amount of cedazuridine and an effective amount of decitabine increases the subject's leukemia-free survival to about 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400%, relative to the leukemia-free survival obtained with BSC measures alone.

In some embodiments, administering an effective amount of cedazuridine and an effective amount of decitabine to a subject with MDS provides the subject with a survival (e.g., overall median survival) of at least about 28, 30, 32, 34, or 36 months, including at least about 28, 30, 31, 32, 33, 34, 35, or 36 months.

In some embodiments, administering an effective amount of cedazuridine and an effective amount of decitabine to a subject with MDS provides the subject with a leukemia-free survival (e.g., median leukemia-free survival) of at least about 26, 28, 30, 32, or 34 months, including at least about 26, 28, 30, 31, 32, 33, 34 months.

Provided according to embodiments of the invention are methods of treating lower-risk MDS in a subject in need thereof. In some embodiments, such methods include administering to the subject an effective amount of cedazuridine and an effective amount of decitabine, thereby treating the lower-risk MDS. In some embodiments, administering an effective amount of cedazuridine and an effective amount of decitabine increases the subject's survival (e.g., overall median survival) to about 130% to about 400% relative to survival obtained by treatment with a hypomethylating agent alone. As such, in some embodiments, administering an effective amount of cedazuridine and an effective amount of decitabine increases the subject's survival to about 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400% or more, or any range defined therebetween, relative to survival obtained by treatment with a hypomethylating agent alone.

In some embodiments, administering an effective amount of cedazuridine and an effective amount of decitabine increases the lower-risk IMDS subject's leukemia-free survival (e.g., median leukemia-free survival) to about 130% to about 400% relative to the leukemia-free survival obtained by treatment with a hypomethylating agent alone. As such, in some embodiments, administering an effective amount of cedazuridine and an effective amount of decitabine increases the subject's leukemia-free survival to about 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400% or more, or any range defined therebetween, relative to the leukemia-free survival obtained by treatment with a hypomethylating agent alone.

In some embodiments, administering an effective amount of cedazuridine and an effective amount of decitabine increases the lower-risk MDS subject's survival to about 200% to about 400% relative to survival obtained when the subject only receives best supportive care (BSC). As such, in some embodiments, administering an effective amount of cedazuridine and an effective amount of decitabine increases the subject's survival to about 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400%, or any range defined therebetween, relative to survival obtained with BSC measures alone.

In some embodiments, administering an effective amount of cedazuridine and an effective amount of decitabine increases the lower-risk MDS subject's leukemia-free survival (e.g., median leukemia-free survival) to about 200% to about 400% relative to the leukemia-free survival obtained when the subject only receives best supportive care (BSC). As such, in some embodiments, administering an effective amount of cedazuridine and an effective amount of decitabine increases the subject's leukemia-free survival to about 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400%, or any range defined therebetween, relative to the leukemia-free survival obtained with BSC measures alone.

In some embodiments, administering an effective amount of cedazuridine and an effective amount of decitabine to a subject with lower-risk MDS provides the subject with a survival (e.g., overall median survival) of at least about 28, 30, 32, 34, or 36 months, including at least about 28, 30, 31, 32, 33, 34, 35, or 36 months.

In some embodiments, administering an effective amount of cedazuridine and an effective amount of decitabine to a subject with lower-risk MDS provides the subject with a leukemia-free survival (e.g., median leukemia-free survival) of at least about 26, 28, 30, 32, or 34 months, including at least about 26, 28, 30, 31, 32, 33, 34 months.

In some method embodiments of the invention, treatment of MDS includes treatment of a lower-risk MDS subject as determined by the International Prognostic Scoring System (IPPS), including Low and Int-1 subgroups. In some method embodiments of the invention, treatment of MDS includes treatment of a lower-risk MDS subject as determined by the International Prognostic Scoring System-Revised (IPPS-R), including the Intermediate, Low, and Very Low subgroups. In some method embodiments of the invention, treatment of MDS includes treatment of a lower-risk MDS subject as determined by the WHO Classification-based Prognostic Scoring System (WPSS), including Intermediate, Low, and Very Low subgroups. In some method embodiments of the invention, treatment of MDS includes treatment of a lower-risk MDS subject as determined by the French-American-British (FAB) criteria.

Provided according to embodiments of the invention are methods of treating chronic myelomonocytic leukemia (CMML) in a subject in need thereof. In some embodiments, such methods include administering to the subject an effective amount of cedazuridine and an effective amount of decitabine, thereby treating the CMML. In some embodiments, administering an effective amount of cedazuridine and an effective amount of decitabine increases the subject's survival (e.g., overall median survival) to about 130% to about 400% relative to survival obtained by treatment with a hypomethylating agent alone. As such, in some embodiments, administering an effective amount of cedazuridine and an effective amount of decitabine increases the subject's survival to about 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400% or more, or any range defined therebetween, relative to survival obtained by treatment with a hypomethylating agent alone.

In some embodiments, administering an effective amount of cedazuridine and an effective amount of decitabine increases the subject's leukemia-free survival (e.g., median leukemia-free survival) to about 130% to about 400% relative to the leukemia-free survival obtained by treatment with a hypomethylating agent alone. As such, in some embodiments, administering an effective amount of cedazuridine and an effective amount of decitabine increases the subject's leukemia-free survival to about 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400% or more, or any range defined therebetween, relative to the leukemia-free survival obtained by treatment with a hypomethylating agent alone.

In some embodiments, administering an effective amount of cedazuridine and an effective amount of decitabine increases the CMML subject's survival to about 200% to about 400% relative to survival obtained when the subject only receives best supportive care (BSC).

As such, in some embodiments, administering an effective amount of cedazuridine and an effective amount of decitabine increases the subject's survival to about 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, or 400%, relative to the survival obtained with BSC measures alone.

In some embodiments, administering an effective amount of cedazuridine and an effective amount of decitabine increases the CMML subject's leukemia-free survival to about 200% to about 400% relative to the leukemia-free survival obtained when the subject only receives best supportive care (BSC). As such, in some embodiments, administering an effective amount of cedazuridine and an effective amount of decitabine increases the subject's leukemia-free survival to about 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, or 400%, relative to the leukemia-free survival obtained with BSC measures alone.

In some embodiments, administering an effective amount of cedazuridine and an effective amount of decitabine to a subject with CMML provides the subject with a survival (e.g., overall median survival) of at least about 28, 30, 32, 34, or 36 months, including at least about 28, 30, 31, 32, 33, 34, 35, or 36 months.

In some embodiments, administering an effective amount of cedazuridine and an effective amount of decitabine to a subject with CMML provides the subject with a leukemia-free survival (e.g., median leukemia-free survival) of at least about 26, 28, 30, 32, or 34 months, including at least about 26, 28, 30, 31, 32, 33, 34 months.

In some method embodiments of the invention, treatment of CMML includes treatment of a CMML subject that meets the French-American-British (FAB) criteria.

Mutations in the TP53 gene (TP53mut) encoding p53 protein in MDS and CMML patients have been characterized as an independent prognostic factor for poor outcome. The present invention has been demonstrated to provide improved outcomes in overall survival and leukemia-free survival for patients with TP53 mutations, either mono-allelic or bi-allelic. The TP53 mutation may be any mutation in the gene that results in decreased TP53 mRNA and/or p53 protein levels or results in decreased p53 activity.

Provided according to embodiments of the invention are methods of treating MDS or CMML in a TP53mut subject in need thereof. In some embodiments, such methods include administering to the subject an effective amount of cedazuridine and an effective amount of decitabine, thereby treating the MDS or CMML. In some embodiments, administering an effective amount of cedazuridine and an effective amount of decitabine increases the subject's survival (e.g., overall median survival) to about 130% to about 400% relative to survival obtained by treatment with a hypomethylating agent alone. As such, in some embodiments, administering an effective amount of cedazuridine and an effective amount of decitabine increases the subject's survival to about 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400% or more, or any range defined therebetween, relative to survival obtained by treatment with a hypomethylating agent alone.

In some embodiments, administering an effective amount of cedazuridine and an effective amount of decitabine increases the TP53mut subject's leukemia-free survival (e.g., median leukemia-free survival) to about 130% to about 400% relative to the leukemia-free survival obtained by treatment with a hypomethylating agent alone. As such, in some embodiments, administering an effective amount of cedazuridine and an effective amount of decitabine increases the subject's leukemia-free survival to about 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400% or more, or any range defined therebetween, relative to the leukemia-free survival obtained by treatment with a hypomethylating agent alone.

In some embodiments, administering an effective amount of cedazuridine and an effective amount of decitabine increases the TP53mut subject's survival to about 200% to about 400% relative to survival obtained when the subject only receives best supportive care (BSC).

As such, in some embodiments, administering an effective amount of cedazuridine and an effective amount of decitabine increases the subject's survival to about 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400%, or any range defined therebetween, relative to survival obtained with BSC measures alone.

In some embodiments, administering an effective amount of cedazuridine and an effective amount of decitabine increases the TP53mut subject's leukemia-free survival (e.g., median leukemia-free survival) to about 200% to about 400% relative to the leukemia-free survival obtained when the subject only receives best supportive care (BSC). As such, in some embodiments, administering an effective amount of cedazuridine and an effective amount of decitabine increases the subject's leukemia-free survival to about 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400%, or any range defined therebetween, relative to the leukemia-free survival obtained with BSC measures alone.

In some embodiments, administering an effective amount of cedazuridine and an effective amount of decitabine to a MDS or CMML subject with a mono-allelic TP53mut provides the subject with a survival (e.g., overall median survival) or leukemia-free survival of at least about 20, 22, 24, 26, 28, 30, 32, 34, or 36 months.

In some embodiments, administering an effective amount of cedazuridine and an effective amount of decitabine to a MDS or CMML subject with a bi-allelic TP53mut provides the subject with a survival (e.g., overall median survival) or leukemia-free survival of at least about 10, 12, 14, 16, 18, or 20 months.

In some embodiments of the invention, administering an effective amount of cedazuridine and an effective amount of decitabine to a subject includes administering to a subject cedazuridine and decitabine in one or more oral dosage forms of the invention. In some embodiments, each oral dosage form is a solid oral dosage form. In some embodiments, a single solid oral dosage form that includes cedazuridine and decitabine is administered. In particular embodiments, provided are methods of administering to a subject an oral dosage form comprising cedazuridine (e.g., 100 mg) and decitabine (e.g., 35 mg) and a pharmaceutically acceptable excipient.

Any administration regimen known to those skilled in the art for regulating the timing and sequence of drug delivery can be used and repeated as necessary to effect treatment in the methods of the invention. For example, the oral dosage forms may be administered 1, 2, 3 or 4 times daily, by a single dose, multiple discrete doses, or continuous infusion. In particular embodiments, at least one solid oral dosage form is administered once daily. In some embodiments, administering at least one solid oral dosage form according to an embodiment of the invention may be performed for one or more weeks per 28-day cycle, e.g., one week, two weeks, three weeks, or four weeks per 28-day cycle. The weeks may be consecutive and/or non-consecutive. In particular embodiments, cedazuridine and decitabine are administered daily for 5 days (days 1-5) of a 28-day cycle.

In some embodiments of the invention, a solid oral dosage form comprising cedazuridine and decitabine is administered to a subject once daily for 1, 2, 3, 4, 5, 6, 7, 8, 9, 10, 11, 12, 13, 14, 15, 16, 17, 18, 19, 20, 21, 22, 23, 24, 25, 26, 27, 28, or more days. In some embodiments, a solid oral dosage form comprising cedazuridine and decitabine is administered to a subject once daily for days 1-5 of a 28-day cycle. In some embodiments, on days 1-5 of a cycle, cedazuridine and decitabine are each administered to the subject daily and on days 6-28 of the cycle, neither cedazuridine nor decitabine is administered. Such solid oral dosage forms may be administered concurrently, sequentially, or at different times during the same day. In particular embodiments, all solid oral dosage forms are administered concurrently or at approximately the same time (e.g., within 5, 10, 15, 20, 25, or 30 minutes).

In some embodiments, a single unit dosage form comprising 35 mg decitabine and 100 mg cedazuridine may be administered daily on each of days 1-5 in the cycle. However, in some embodiments, the doses of cedazuridine and decitabine may be varied. This may be appropriate, for example, when it is a second or later cycle for the subject. More information regarding possible dosing schedules and administration for cedazuridine and decitabine may be found in the Inqovi® prescribing information and at www.inqovi.com.

In some embodiments, a time period of 0 to 31 days or more (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more) may pass between multiple cycles of the present invention. The time period of no treatment may be desirable to allow a subject (e.g., a human patient) of the present invention to have adequate health to continue treatment. The time period between treatment cycles can be determined by a physician using standard techniques in the art and may be determined individually on a per-subject basis, for example, as based on adequate blood count, e.g., adequate lack of neutropenia (e.g., absolute neutrophil count (ANC) in the subject of at least or greater than 0.5×10⁹ cells/L), and may be adjusted over the course of treatment based on the judgement of the administering physician. In some embodiments, the time period between treatment cycles may be minimal, e.g., no time period, e.g., immediately starting on the next 28-day time period. In some embodiments, the time period between treatment cycles may be 1 week, 2 weeks, 3 weeks, 4 weeks, 5 weeks, 6 weeks, or more.

In some embodiments, the administration regimen may include pretreatment and/or co-administration with at least one additional therapeutic agent. In such case, the decitabine and cedazuridine may be administered with the at least one additional therapeutic agent simultaneously, separately, or sequentially. The additional therapeutic agent may also be included within the one or more solid oral dosage forms. As used herein, the term “additional therapeutic agents” includes, but is not limited to, chemotherapeutic agents and immunomodulatory agents.

Examples of chemotherapeutic agents include without limitation: alkylating agents (e.g., which may include doxorubicin, cyclophosphamide, estramustine, carmustine, mitomycin, bleomycin and the like); antimetabolites (e.g., which may include 5-fluoro-uracil, capecitabine, gemcitabine, nelarabine, fludarabine, methotrexate and the like); platinating agents (e.g., which may include cisplatin, oxaliplatin, carboplatin and the like); topoisomerase inhibitors (e.g., which may include topotecan, irinotecan, etoposide and the like); tubulin agents (e.g., which may include paclitaxel, docetaxel, vinorelbine, vinblastine, vincristine, other taxanes, epothilones, and the like); signaling inhibitors (e.g., kinase inhibitors, antibodies, farnesyltransferase inhibitors, and the like); and other hormonal or chemotherapeutic agents (e.g., tamoxifen, anti-mitotic agents such as polo-like kinase inhibitors or aurora kinase inhibitors, and the like). Examples of immunomodulatory agents include but not limited to CTLA-4, PD-1, and PDL-1 antibodies or inhibitors, lenalidomide, and the like.

Administering to a subject in need thereof of a combination of decitabine and cedazuridine (e.g., in a solid oral dosage form) according to an embodiment of the invention may provide multiple beneficial responses to the subject. For example, in some embodiments, the administering reduces DNA methylation in the subject by at least 5% (e.g., at least 5, 6, 7, 8, 9, 10, 11, 12, 13, 14, or 15% or more or any value or range therein) as compared to a control measurement, e.g., as compared to DNA methylation in the subject prior to the administering (e.g., subject “baseline” DNA methylation). DNA methylation in the subject may be quantitatively and/or qualitatively evaluated by any standard technique in the art, e.g., as measured by a marker of relative global methylation as compared to a control, e.g., as measured by Long interspersed element-1 (LINE-1) methylation as compared to a control. For example, in some embodiments, the administering reduces LINE-1 methylation in the subject by at least 5% (e.g., at least 5, 6, 7, 8, 9, 10, 11, 12, 13, 14, or 15% or more) as compared to a control measurement, e.g., as compared to LINE-1 methylation in the subject prior to the administering (e.g., subject baseline LINE-1 methylation). For example, in some embodiments, the administering may reduce LINE-1 methylation in the subject by at least 5%, at least 8%, at least 10% or at least 15% or more. In some embodiments, the administering may reduce LINE-1 methylation in the subject by about 5% to about 20%, about 6% to about 15%, or by about 8% to about 10%.

In some embodiments, administering decitabine and cedazuridine to the subject may reduce absolute neutrophil count (ANC) in the subject to less than 0.5×10⁹ cells/L of blood for no more than two, three, or four weeks (e.g., no more than 28, 27, 26, 25, 24, 23, 22, 21, 20, 19, 18, 17, 16, 15, 14, 13, 12, 11, 10, 9, 8, 7, 6, 5, 4, 3, 2, or 1 consecutive days or any value or range therein) following a 28-day cycle. In some embodiments, administering decitabine and cedazuridine to the subject reduces absolute neutrophil count (ANC) in the subject to less than 0.5×10⁹ cells/L of blood for no more than two, three, or four weeks during treatment (e.g., between multiple, repeated 28-day cycles).

In some embodiments, administering decitabine and cedazuridine to the subject expands hemoglobin F-expressing cells (i.e., F cells) by at least 5% (e.g., at least 5, 6, 7, 8, 9, 10, 15, 20, 25, or 30% or more), optionally as measured by % F cells/erythrocytes per sample (e.g., in a patient blood sample) as compared to a “baseline” control % F cells/erythrocytes (e.g., as compared to % F cells/erythrocytes of the patient prior to treatment, e.g., as compared to the average % F cells/erythrocytes of a patient population not undergoing treatment (e.g., a healthy patient population)). For example, in some embodiments, administering decitabine and cedazuridine to the subject may expand % F cells in the subject by at least 5%, at least 8%, at least 10%, at least 15%, or at least 23% or more as compared to a baseline control. In some embodiments, the administering may expand % F cells in the subject by about 5% to about 30%, about 6% to about 24%, or by about 8% to about 20% as compared to a baseline control.

In some embodiments, administering decitabine and cedazuridine to the subject expands F cells to a total amount of at least 10% to at least 30% or more of total erythrocytes (e.g., at least 15%, 16%, 17%, 18%, 19%, 20%, 21%, 22%, 23%, 24%, 25%, 26%, 27%, 28%, 29%, or 30% or more F cells/erythrocytes or any value or range therein) per sample (e.g., in a patient blood sample). For example, in some embodiments, administering decitabine and cedazuridine to the subject may expand F cells to a total amount of at least 15%, at least 20%, at least 23%, at least 35% or more of total erythrocytes in a sample. In some embodiments, administering decitabine and cedazuridine to the subject may expand F cells to a total amount of about 15% to about 30%, about 18% to about 25%, or about 15% to about 35%, of total erythrocytes in a sample.

In some embodiments of the invention, the subject is aged 75 years or older. In particular embodiments, the subject is aged 18 years or older and ineligible for induction chemotherapy due to one or more comorbidities. Such comorbidities include, for example, (i) a baseline Eastern Cooperative Oncology Group (ECOG) performance status of 2 or 3, (ii) severe cardiac disorder (e.g., congestive heart failure requiring treatment, ejection fraction less than or equal to 50%, or chronic stable angina), (iii) severe pulmonary disorder (e.g., diffusing lung capacity for carbon monoxide DLCO less than or equal to 65% or forced expiratory volume in 1 second (FEV1) of less than or equal to 65%, (iv) creatinine clearance greater than or equal to 30 mL/min and less than 45 mL/min, and/or (v) moderate hepatic impairment with total bilirubin greater than 1.5 and less than or equal to 3.0×the upper limit of normal (ULN).

Dose levels, mode of administration, and administration regimen may be modified by those skilled in the art using known techniques as judged necessary for the subject (e.g., the patient).

### Oral Dosage Formulations

The methods described herein may use any form of decitabine and cedazuridine and/or a pharmaceutically acceptable salt of either compound. As used herein, decitabine and cedazuridine, and their pharmaceutically acceptable salts, may be referred to collectively as “therapeutic agents.” Additionally, when a therapeutic agent is referenced, it is to be understood that the therapeutic agent's pharmaceutically acceptable salts are also included in this reference. In some embodiments, the therapeutic agents are administered in oral dosage forms, such as solid oral dosage forms. Such oral dosage forms may include each therapeutic agent separately or may be include a combination dosage form. In particular embodiments, subjects are administered a solid oral dosage form that includes decitabine and cedazuridine.

In some embodiments, the one or more of the oral dosage forms is a solid oral dosage form, such as a solid oral unit dosage form. The term “solid oral dosage form” means that the pharmaceutical compositions are in solid form and are formulated for oral administration. Any suitable solid oral dosage form may be used. Examples of solid oral dosage forms according to embodiments of the invention include tablets (for example, those targeted for buccal, sublingual and systemic absorption), caplets, boluses, powders, granules, pastes for application to the tongue, capsules including hard gelatin capsules and soft gelatin capsules, mouth sprays, troches, lozenges, and pellets. The pharmaceutical compositions may be formulated for immediate, sustained, or controlled release.

A number of possible oral dosage forms could be used in the methods of the invention. For example, decitabine may be present in an oral dosage form in a range of about 10 mg to about 100 mg, about 20 mg to about 45 mg, or about 30 mg to about 40 mg (e.g., about 10, 20, 25, 30, 35, 40, 45, 50, 55, 60, 65, 70, 75, 80, 90, or 100 mg). As another example, cedazuridine may be present in an oral dosage form in a range of about 10 mg to about 150 mg, about 70 mg to 120 mg, or about 90 mg to about 110 mg (e.g., about 50, 60, 70, 80, 85, 90, 95, 100, 105, 110, 115, 120, 125, 130, 140, or 150 mg). However, in some embodiments of the invention, one solid oral dosage form is a unit dosage form that comprises about 35 mg decitabine. In some embodiments of the invention, one solid oral dosage form is unit dosage form that comprises about 100 mg of cedazuridine. Furthermore, in some embodiments, one solid oral dosage form is a unit dosage form (e.g., a fixed-dose combination) that comprises about 35 mg decitabine and about 100 mg of cedazuridine. In some embodiments of the invention, one unit dosage form comprises about 35 mg decitabine and about 100 mg of cedazuridine and at least one pharmaceutically acceptable excipient.

Pharmaceutically acceptable excipients are well known in the pharmaceutical arts and are described, for example, in Remington's Pharmaceutical Sciences, Mack Publishing Co., Easton, Pa (e.g., 20th Ed., 2000), and Handbook of Pharmaceutical Excipients, American Pharmaceutical Association, Washington, D.C., (e.g., 1st, 2nd and 3rd Eds., 1986, 1994 and 2000, respectively). As will be known to those skilled in the art, excipients may provide a variety of functions and may be described as, e.g., wetting agents, buffering agents, suspending agents, diluents, binders, lubricating agents, glidants, emulsifiers, disintegrants, absorbents, preservatives, surfactants, colorants, flavorants, and sweeteners. Examples of pharmaceutically acceptable excipients include without limitation: (1) sugars, such as lactose, glucose and sucrose; (2) starches, such as corn starch and potato starch; (3) cellulose and its derivatives, such as sodium carboxymethyl cellulose, ethyl cellulose, cellulose acetate, hydroxypropylmethylcellulose (hypromellose), and hydroxypropylcellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Examples of diluents include lactose, lactose monohydrate, cellulose, microcrystalline cellulose, sorbitol, dibasic calcium phosphate dehydrate, and calcium sulfate dehydrate. Examples of binders include gelatin, glucose, lactose, cellulose, methyl cellulose, ethyl cellulose, hydroxypropyl methyl cellulose (hypromellose), hydroxypropyl cellulose, starch, poly vinyl pyrrolidone, sodium alginate, carboxymethylcellulose, and acacia. Examples of disintegrants include croscarmellose sodium, crospovidone, sodium starch glycollate, and starch. Examples of glidants include colloidal silicon dioxide, cornstarch and talc. Examples of lubricants include stearic acid, magnesium stearate, calcium stearate, talc, paraffin, sodium lauryl sulphate, sodium benzoate, and polyethylene glycol.

In some embodiments, a solid oral dosage form includes one or more of a diluent, binder, disintegrant, glidant, and lubricant. In some embodiments, a solid oral dosage form includes a diluent, binder, disintegrant, glidant, and lubricant. In particular embodiments of the invention, the solid oral dosage form includes decitabine and/or cedazuridine and the following excipients: lactose monohydrate as a diluent; hydroxypropyl methylcellulose as a binder; croscarmellose sodium as a disintegrant; colloidal silicon dioxide as a glidant; and magnesium stearate as a lubricant. In some embodiments of the invention, such components are formed into a tablet. In some embodiments, the tablet is an immediate release tablet. Additionally, in particular embodiments, the tablet is coated with a film, which may be colored. Any pharmaceutically acceptable coating may be used but, in some embodiments, the tablet is coated with an Opadry® coating.

In some embodiments, cedazuridine is present in a solid oral dosage form in an amount of about 17-22% w/w, e.g., about 17.0, 17.2, 17.4, 17.6, 17.8, 18.0, 18.2, 18.4, 18.6, 18.8, 19.0, 19.2, 19.4, 19.6, 19.8, 20.0, 20.2, 20.4, 20.6, 20.8, 21.0, 21.2, 21.4, 21.6, 21.8, or 22.0% w/w or any range therein, e.g., about 19.42% w/w. In some embodiments, decitabine is present in the solid oral dosage form in an amount of about 4-8% w/w, e.g., about 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, or 8.0% w/w or any range therein, e.g., about 6.8% w/w. In some embodiments, the diluent (e.g., lactose monohydrate) is present in the solid oral dosage form in an amount of about 55-70% w/w, e.g., about 55, 56, 57, 58, 59, 60, 61, 62, 63, 64, 65, 66, 67, 68, 69, or 70% w/w or any range therein, e.g., about 62.62% w/w. In some embodiments, the binder (e.g., hypromellose) is present in the solid oral dosage form in an amount of about 1-3% w/w, e.g., about 1.0, 1.2, 1.4, 1.6, 1.8, 2.0, 2.2, 2.4, 2.6, 2.8, or 3.0% w/w or any range therein, e.g., about 1.94% w/w. In some embodiments, the disintegrant (e.g., croscarmellose sodium) is present in the solid oral dosage form in an amount of about 3-7% w/w, e.g., about 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, or 7.0% w/w or any range therein, e.g., about 4.85% w/w. In some embodiments, the glidant (e.g., colloidal silicon dioxide) is present in the solid oral dosage form in an amount of about 0.5-2% w/w, e.g., about 0.5, 0.6, 0.7, 0.8, 0.9, 1.0, 1.1, 1.2, 1.3, 1.4, 1.5, 1.6, 1.7, 1.8, 1.9, or 2.0% w/w or any range therein, e.g., about 0.97% w/w. In some embodiments, the lubricant (e.g., magnesium stearate) is present in the solid oral dosage form in an amount of about 0.1-2% w/w, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0% w/w or any range therein, e.g., about 0.49% w/w.

In some embodiments, the solid oral dosage form comprises the components listed in Table 1.

TABLE 1

| Component | Function | Composition (% w/w) |
|---|---|---|
| Cedazuridine | Active | 17-22 |
| Decitabine | Active | 4-8 |
| Lactose monohydrate | Diluent | 55-70 |
| Hypromellose | Binder | 1-3 |
| Croscarmellose sodium | Disintegrant | 3-7 |
| Colloidal silicon dioxide | Glidant | 0.5-2 |
| Magnesium stearate | Lubricant | 0.1-2 |

In some embodiments, the solid oral dosage form comprises the components listed in Table 2.

TABLE 2

| Component | Function | Composition (% w/w) |
|---|---|---|
| Cedazuridine | Active | 19.42 |
| Decitabine | Active | 6.80 |
| Lactose monohydrate | Diluent | 62.62 |
| Hypromellose | Binder | 1.94 |
| Croscarmellose sodium | Disintegrant | 4.85 |
| Colloidal silicon dioxide | Glidant | 0.97 |
| Magnesium stearate | Lubricant | 0.49 |
| Total Core Tablet | | 97.09 |
| Coating | Film Coat | 2.91 |
| Total Coated Tablet | | 100.0 |

More information regarding compositions and formulations for cedazuridine and decitabine may be found in the Inqovi® prescribing information and at www.inqovi.com.

Pharmaceutical compositions of the invention can be prepared using known materials and techniques, which may include, but are not limited to, mixing and/or blending decitabine and cedazuridine with the pharmaceutically acceptable excipients.

In some embodiments of the invention, the methods of the invention are performed using a kit comprising at least one unit dosage form comprising decitabine and at least one unit dosage form comprising cedazuridine. In particular embodiments, a kit comprises at least one unit dosage form comprising decitabine and cedazuridine. In some embodiments, the kit provides one unit dosage form that includes about 35 mg decitabine and about 100 mg of cedazuridine and at least one pharmaceutically acceptable excipient. Other unit dosage forms may be used and may vary depending on availability. The daily dose for cedazuridine and/or decitabine may require more than one unit dosage form per day.

A kit may include the solid oral dosage forms for one day's dose of each therapeutic agent (e.g., one tablet comprising 35 mg decitabine and 100 mg cedazuridine). A kit may also include unit dosage forms for more than one day of the cycle (e.g., a week) or for the full cycle. As such, in some embodiments of the invention, a kit may include 1, 2, 3, 4, 5, 6, 7, 8, 9, 10, 11, 12, 13, 14, 15, 16, 17, 18, 19, 20, 21, 22, 23, 24, 25, 26, 27, 28 or more solid oral dosage forms comprising cedazuridine and decitabine according to an embodiment of the invention. In some embodiments, a kit may include 5 solid oral dosage forms comprising cedazuridine and decitabine (for days 1-5 of the cycle).

The kit may further comprise a container and/or a package suitable for commercial sale. The container can be in any conventional shape or form known in the art which is made of a pharmaceutically acceptable material, such as a paper or cardboard box, a glass or plastic bottle or jar, a re-sealable bag, or a blister pack with individual dosages for pressing out of the pack according to a therapeutic schedule. More than one container can be used together in a single package. For example, tablets may be contained in a blister pack which is in turn contained within a box. In some embodiments, the container is a bottle, e.g., a 30-cc white high-density polyethylene bottles containing unit dosage forms (e.g., about 5-unit dosage forms). The bottle may further contain desiccant, e.g., silica desiccant canisters. In some embodiments, the container is a blister pack, e.g., formed by aluminum foil on foil lidding containing one tablet per cavity. The blister packs may be present in a carton.

The kit may further comprise information. The information may be provided on a readable medium. The readable medium may comprise a label. The information may be directed towards a physician, pharmacist, or patient. The information may indicate that the unit dosage form may cause one or more adverse effects. The information may comprise instructions for administering the unit dosage form, such as in a manner described herein. These instructions may be provided in a variety of ways.

The information can be associated with the container, for example, by being: written on a label (e.g., the prescription label or a separate label) adhesively affixed to a container; included inside a container as a written package insert; applied directly to the container such as being printed on the wall of a box or blister pack; or attached as by being tied or taped, for example as an instructional card affixed to the neck of a bottle via a string, cord or other line, lanyard or tether type device.

It will be apparent to those skilled in the art that specific embodiments of the present invention may be directed to one, some or all of the above-indicated aspects as well as other aspects, and may encompass one, some or all of the above- and below-indicated embodiments, as well as other embodiments.

Other than in the working examples, or where otherwise indicated, all numbers expressing quantities of ingredients, reaction conditions, and so forth used in the specification and claims are to be understood as being modified by the term “about”. Accordingly, unless indicated to the contrary, such numbers are approximations that may vary depending upon the-desired properties sought to be obtained by the present invention. At the very least, and not as an attempt to limit the application of the doctrine of equivalents to the scope of the claims, each numerical parameter should be construed in light of the number of significant digits and ordinary rounding techniques.

While the numerical ranges and parameters setting forth the broad scope of the invention are approximations, the numerical values set forth in the working examples are reported as precisely as possible. Any numerical value, however, inherently contains certain errors necessarily resulting from the standard deviation found in their respective testing measurements.

Having described the present invention, the same will be explained in greater detail in the following examples, which are included herein for illustration purposes only, and which are not intended to be limiting to the invention.

### Example 1: Oral Decitabine/Cedazuridine Vs. IV Decitabine in MDS Including Lower-Risk MDS and CMML (Phase 3 Clinical Trial)

Myelodysplastic syndromes (MDS) comprise a spectrum of myeloid malignancies characterized by the acquisition of somatic mutations, ineffective hematopoiesis, peripheral blood cytopenia, and a high risk of progression to acute myeloid leukemia (AML).1 While the only known curative treatment is allogeneic hematopoietic stem cell transplantation (HSCT), many patients are ineligible for HSCT, and the DNA methyltransferase inhibitors (DNMTi's) azacitidine and decitabine are the standard of care as disease-modifying therapies for patients with higher risk MDS and for some patients with lower risk MDS.² Until recently, both DNMTi's approved for use in MDS were administered parenterally, requiring patients to come to a treatment center daily for 5 or 7 consecutive days of every 28-day treatment cycle. As ongoing treatment is required to sustain response,³ these parenteral regimens represent a substantial burden for the older adult population affected by MDS (median age of 73 at diagnosis).² Patients may require transportation to appointments and support during and after therapy⁴⁻⁶ and the existing infection risk was worsened during the SARS-CoV-2 pandemic.^{7,8}

The oral bioavailability of both DNMTi's is low due to first-pass elimination by the enzyme cytidine deaminase found in the gut and liver, and the development of an orally available DNMTi has been a goal since the introduction of this drug class.^{9,10} Blocking cytidine deaminase is one strategy for increasing oral systemic exposure of decitabine to therapeutic plasma concentrations of decitabine. Cedazuridine is a synthetic nucleoside analog derived from a competitive cytidine deaminase inhibitor. A phase 1, dose-finding study identified the doses of oral decitabine and cedazuridine (30-40 and 100 mg, respectively) that produced systemic exposure closest to that of the standard 5-day regimen for IV decitabine in MDS.¹¹ A phase 2, randomized, crossover trial established that oral decitabine 35 mg and oral cedazuridine 100 mg, given simultaneously as separate capsules and then as a fixed-dose combination, generated decitabine exposure, DNA demethylation, and safety comparable to those of IV decitabine with clinical responses similar to those previously reported for IV decitabine.^{12,13} Here, the results of a phase 3 trial are presented comparing intrapatient total systemic exposure of oral decitabine/cedazuridine and IV decitabine over 5 days. Pharmacodynamics (DNA demethylation), clinical response, survival, and safety were also assessed.

### Methods

Trial design and oversight: Eligible patients were randomized in a 1:1 ratio to one of two sequences for the first two 28-day treatment cycles administered: oral decitabine/cedazuridine (1 tablet once daily containing decitabine 35 mg and cedazuridine 100 mg as a fixed-dose combination) in cycle 1 and IV decitabine (20 mg/m²/d by continuous 1-hour IV infusion)¹⁴ in cycle 2 (sequence A), or the reverse (sequence B). Each treatment was given for the first 5 consecutive days of a 28-day cycle. After cycle 2, patients received treatment with oral decitabine/cedazuridine for the first 5 days of each successive 28-day cycle until disease progression, unacceptable toxicity, or treatment discontinuation for other reasons. Consistent with standard clinical care with parenteral DNMTi, cycles could be delayed or doses reduced if judged necessary for hematologic or nonhematologic recovery.

The protocol is available at NEJM.org. Patients who benefited from oral decitabine/cedazuridine were offered the option to enter an extension study assessing survival and long-term safety, which are described here (ClinicalTrials.gov Identifier: NCT04093570).

An institutional review board or independent ethics committee at each of the 37 participating study centers in Canada (7 sites) and the United States (30 sites) approved the protocol and amendments. The study was conducted in accordance with the protocol, the International Council for Harmonisation Good Clinical Practice guidelines, and applicable local requirements. Each patient provided informed consent. Clinical response was assessed by an independent review committee.

Eligibility: Patients aged ≥18 years who were candidates to receive IV decitabine (all French-American-British subtypes of previously treated or untreated MDS, chronic myelomonocytic leukemia which scored intermediate-1 or -2, or high-risk on the International Prognostic Scoring System),¹⁴ with Eastern Cooperative Oncology Group performance status 0 or 1, and a life expectancy ≥3 months, were eligible for inclusion. One prior treatment cycle with decitabine or azacitidine was permitted. Patients who had previously received more than one prior cycle of DNMTi were excluded.

Response criteria and endpoints: The primary endpoint was the comparison of decitabine total exposure over 5 days between oral decitabine/cedazuridine and IV decitabine for cycles 1 and 2, measured as area under the curve (AUC). Secondary parameters included other pharmacokinetic measurements; pharmacodynamics (DNA demethylation of oral decitabine/cedazuridine vs IV decitabine, measured by long interspersed nuclear element-1 [% LINE-1] methylation analysis)¹⁵; clinical response (complete response [CR], marrow CR, partial response, and hematologic improvement) based on International Working Group 2006 MDS response criteria¹⁶; red blood cell (RBC) or platelet transfusion independence (defined as ≥56 consecutive days of transfusion independence after first study treatment dose among patients who were transfusion dependent at baseline); leukemia-free and overall survival (LFS and OS, respectively); and safety.

Pharmacokinetic and pharmacodynamic endpoints were assessed based on data from cycles 1 and 2 only, after all evaluable patients had completed both cycles. Efficacy and safety endpoints were calculated using all data through final cutoff. Final data cutoff occurred after all patients completed ≥6 months of follow-up or had permanently discontinued treatment, whichever came first.

Statistical analysis: The primary analysis consisted of two one-sided equivalence tests for the geometric least-squares mean (LSM) ratio of decitabine 5-day AUC for 0-24 hours (AUC₀₋₂₄) between oral decitabine/cedazuridine and IV decitabine. A total of 118 patients was planned for this analysis to provide 90% power at the statistical significance level of 0.05, presuming the true ratio of geometric means is 1.0, the intrapatient coefficient of variation (CV) on an unlogged scale is 0.55, and the 90% confidence interval (CI) equivalence limits for the ratio of geometric means are 0.8 and 1.25. Assuming that 10% of patients would not be evaluable, 132 patients were planned to be randomized. The CV of 0.55 was chosen as a conservative value based on a prior study estimating the intrapatient CV as 0.5.¹² The treatments would be considered equivalent if the 2-sided 90% CI of the geometric LSM ratio was within the prespecified range of 80%-125%.

All statistical tests and CIs were two-sided, with an a of 0.05. The SAS©9.4 statistical package (SAS Institute Inc., Cary, NC) was used for the analysis. Pharmacokinetic analyses were performed with Phoenix© WinNonlin© 7.0 (or higher; Certara, L. P, Princeton, NJ).

The primary endpoint analysis was based on data from patients who received the full treatment dose in cycles 1 and 2, and had decitabine daily AUC₀₋₂₄ for both oral decitabine/cedazuridine and IV decitabine (i.e., paired cycles). For oral therapy, treatment had to occur within 3 hours of intended dosing time, without vomiting within 6 hours of dosing. At least one of either day 1 or 5 decitabine AUC₀₋₂₄ measurement during the IV decitabine cycle was required to be evaluable. Peak decitabine plasma concentrations were detected after 1 hour following both oral and IV therapy, except on day 5 after IV treatment when they were attained earlier at 30 minutes after treatment. Descriptive statistics were calculated for pharmacokinetic values.

Analysis of variance was performed on natural log-transformed 5-day decitabine AUC₀₋₂₄ for oral decitabine/cedazuridine compared with IV decitabine. Effects of the sequence and cycle were evaluated.

### Results

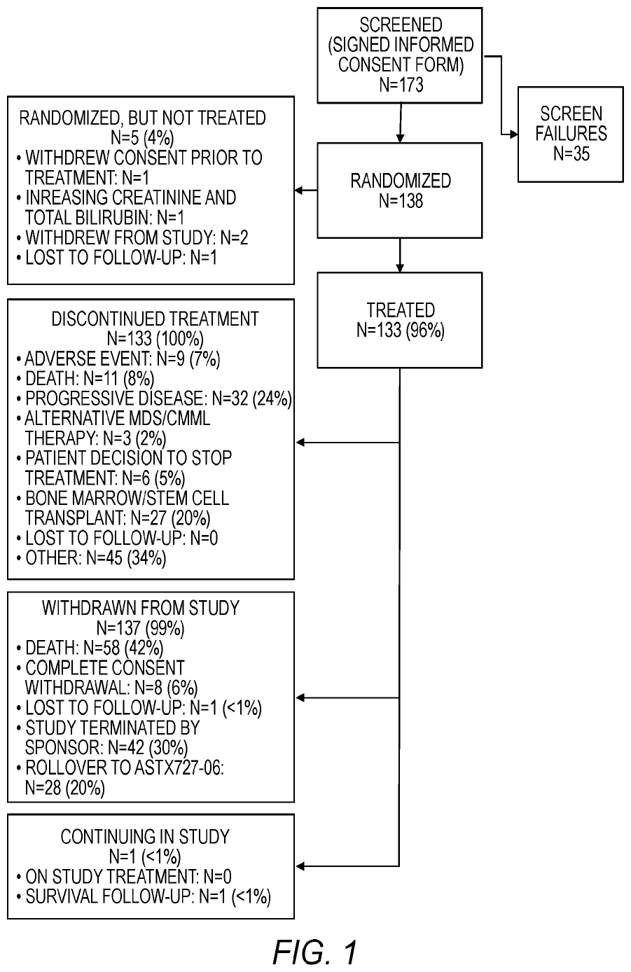

Patients and treatment: In all, 173 patients were screened and 138 were randomized 1:1 between the two treatment sequences (FIG. 1). Five patients received no treatment, and 133 were included in the efficacy and safety analyses. Most treated patients (n=110) were enrolled from the US-based centers; the remaining 23 came from centers in Canada. Median duration of follow-up was 966 days (˜2.6 years; range 868-1208 days).

Most patients (88%) had MDS (Table 1). Baseline characteristics were generally balanced between the treatment sequences. Sequence A (oral therapy first) had a lower proportion of patients with CMML, a higher proportion with higher risk MDS, a higher proportion with low baseline neutrophils, and a lower proportion with high baseline neutrophils.

TABLE 1
Baseline Characteristics.*

| | Sequence A | | Sequence B | | Total | |
|---|---|---|---|---|---|---|
| | (n = 66) | | (n = 67) | | (n = 133) | |
| Age, y | | | | | | |
| Mean (SD) | 68.7 | (10.22) | 70.7 | (8.40) | 69.7 | (9.37) |
| Median (range) | 70.0 | (44, 85) | 72.0 | (49, 88) | 71.0 | (44, 88) |
| Male sex, n (%) | 42 | (64) | 45 | (67) | 87 | (65) |
| Race, n (%) | | | | | | |
| White | 60 | (91) | 61 | (91) | 121 | (91) |
| Black | 1 | (2) | 3 | (4) | 4 | (3) |
| Asian | 2 | (3) | 1 | (1) | 3 | (2) |
| Not reported | 3 | (5) | 2 | (3) | 5 | (4) |
| Median weight, lb (range) | 174.6 | (99.2, 17.4) | 187.0 | (111.3, 60.4) | 183.2 | (99.2, 127.7) |
| Mean BSA, m² (SD) | 1.96 | (0.26)*^{†} | 2.00 | (0.25) | 1.98 | (0.26)^{†‡} |
| Disease and MDS IPSS | | | | | | |
| category, n (%) | | | | | | |
| MDS | 61 | (92) | 56 | (84) | 117 | (88) |
| Low risk^{‡§} | 3 | (5) | 2 | (3) | 5 | (4) |
| Intermediate-1 | 30 | (45) | 34 | (51) | 64 | (48) |
| Intermediate-2 | 14 | (21) | 13 | (19) | 27 | (20) |
| High risk | 14 | (21) | 7 | (10) | 21 | (16) |
| CMML | 5 | (8) | 11 | (16) | 16 | (12) |
| ECOG performance status, | | | | | | |
| n (%) | | | | | | |
| 0 | 25 | (38) | 30 | (45) | 55 | (41) |
| 1 | 41 | (62) | 37 | (55) | 78 | (59) |
| Cytogenetic risk | | | | | | |
| classification, n (%) | | | | | | |
| Poor | 16 | (24) | 17 | (25) | 33 | (25) |
| Intermediate | 13 | (20) | 19 | (28) | 32 | (24) |
| Better | 30 | (45) | 26 | (39) | 56 | (42) |
| Not evaluable/missing | 7 | (11) | 5 | (7) | 12 | (9) |
| >5% bone marrow blasts, n | 29 | (45) | 27 | (40) | 56 | (42) |
| (%) | | | | | | |
| Prior DNMTi, n (%) | | | | | | |
| Azacitidine | 3 | (5) | 3 | (4) | 6 | (5) |
| Decitabine | 3 | (5) | 1 | (1) | 4 | (3) |
| RBC transfusion | 27 | (41) | 27 | (40) | 54 | (41) |
| dependent, n (%) | | | | | | |
| Platelet transfusion | 6 | (9) | 6 | (9) | 12 | (9) |
| dependent, n (%) | | | | | | |
| Median hemoglobin, g/dL | 9.18 | (7.15, 13.9) | 8.75 | (6.4, 14.7) | 8.90 | (6.4, 14.65) |
| (range) | | | | | | |
| <8.0 g/dL, n (%) | 11 | (17) | 16 | (24) | 27 | (20) |
| 8.0-<10.0 g/dL, n (%) | 31 | (47) | 31 | (46) | 62 | (47) |
| 10.0-<11.0 g/dL, n (%) | 14 | (21) | 7 | (10) | 21 | (16) |
| ≥11.0 g/dL, n (%) | 10 | (15) | 13 | (19) | 23 | (17) |
| Median neutrophils, μL, n | 1230 | (50, 9050) | 1485 | (100, 7470) | 1370 | (50, 9050) |
| (%) | | | 15 | (22) | | |
| <500 μ/L, n (%) | 12 | (18) | 6 | (9) | 27 | (20) |
| 500-<1000 μ/L, n (%) | 16 | (24) | 14 | (21) | 22 | (17) |
| 1000-1500 μ/L, n (%) | 13 | (20) | 32 | (48) | 27 | (20) |
| >1500-μ/L, n (%) | 25 | (38) | | | 57 | (43) |
| Median platelets, 10³/μL | 71.8 | (5-530) | 76.0 | (10-730) | 72.5 | (5-703) |
| (range) | | | | | | |
| <25 10³/μL, n (%) | 8 | (12) | 7 | (10) | 15 | (11) |
| 25-<50 10³/μL, n (%) | 14 | (21) | 12 | (18) | 26 | (20) |
| 50-<75 10³/μL, n (%) | 13 | (20) | 14 | (21) | 27 | (20) |
| 75-<100 10³/μL, n (%) | 4 | (6) | 9 | (13) | 13 | (10) |
| ≥100 10³/μL, n (%) | 27 | (41) | 25 | (37) | 52 | (39) |

*Weight SI conversion factor: conventional units × 0.453592; hemoglobin SI conversion factor, conventional units × 0.1; neutrophil conversion factor, conventional units × 0.001; platelet conversion factor, conventional units × 1.
^{†}n = 65.
^{‡}n = 132.
^{§}Patients with low-risk myelodysplastic syndromes (MDS) were eligible according to French-American-British classification criteria per the decitabine labeling.¹⁴
BSA, body surface area; CMML, chronic myelomonocytic leukemia; ECOG, Eastern Cooperative Oncology Group; DNMTi, DNA methyltransferase inhibitor; IPSS, International Prognostic Scoring System; RBC, red blood cells; SD, standard deviation.

Most patients (n=129 [97%]) received at least two treatment cycles. Patients received a median of nine treatment cycles (range 1-28). Most patients (n=100 [75%]) had at least one dose delayed; more than half (n=72 [54%]) had at least one dose-reduced cycle. Median treatment duration was 8.18 months (range 0.2-30.9), with 59% of patients treated for ≥6 months and 39% of patients treated for >12 months. The most common single reason for treatment discontinuation was undergoing hematopoietic cell transplantation (20%; FIG. 1). In all, 123 of the 133 treated patients had paired PK samples and were included in the primary endpoint analysis. Reasons for exclusion include sample integrity or data quality issues (n=2 and n=5, respectively; e.g., samples collected from the second lumen of the IV line) and receiving only one treatment cycle (IV, n=3).

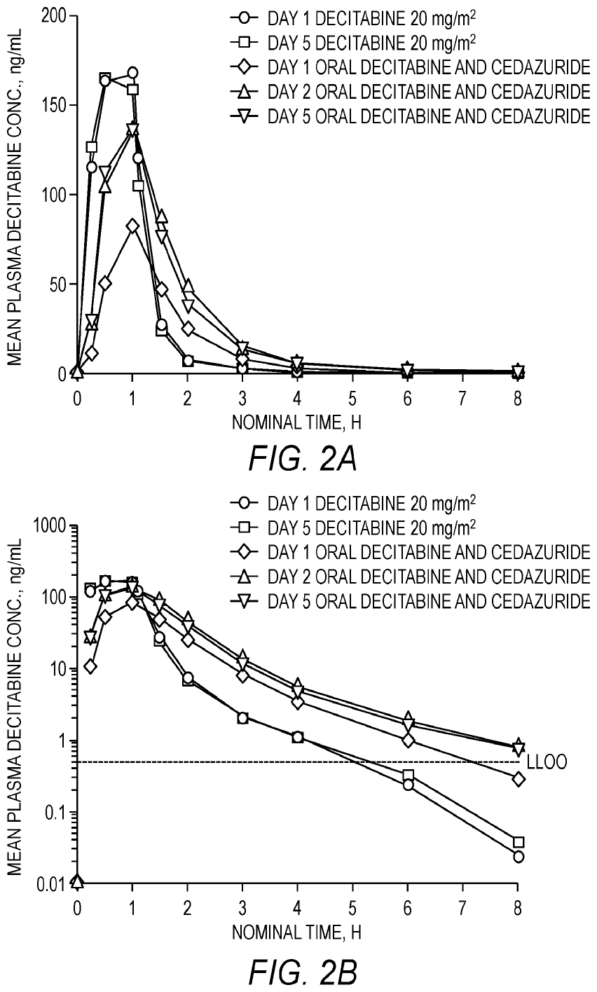

Pharmacokinetics and pharmacodynamics: The geometric LSM ratio of decitabine 5-day AUC₀₋₂₄ between oral decitabine/cedazuridine and IV decitabine was 98.9% (90% CI 92.7%, 105.6%), which was within the prespecified range of 80%-125% (Table 2). Secondary pharmacokinetic parameters support these findings (Table S2). Peak concentrations were higher after IV vs oral administration (FIG. 2A). Plasma concentrations remained above the lower limit of quantification for up to 4 hours in most patients after IV administration and for up to 6 hours in most patients following oral administration (FIG. 2B).

TABLE 2
Pharmacokinetics: 5-Day Decitabine AUC₀₋₂₄ for Oral and IV Formulations.

| | | 5-Day Decitabine AUC₀₋₂₄ | | | |
|---|---|---|---|---|---|
| | | LSM, h × ng/mL (n) | | | |
| | | IV | | Ratio, % | Intrapatient |
| | | Decitabine | Oral D/C | (90% CI)* | CV, % |
| Primary | Paired^{†} | 864.94 (123) | 855.69 (123) | 98.93 (92.66, 105.6) | 31.7 |
| endpoint | | | | | |
| Sensitivity | Unpaired | 865.82 (131) | 848.40 (128) | 97.99 (91.84, 104.5) | 32.2 |
| | Paired^{‡} | 869.96 (128) | 850.32 (128) | 97.74 (91.58, 104.3) | 32.2 |

*Ratio (%) of oral/IV geometric least-squares means (LSMs).
^{†}Excludes data from some patients due to data confidence/quality issues.
^{‡}Includes data excluded from primary endpoint for data confidence/quality issues.
AUC₀₋₂₄, area under curve over last 24-hour dosing interval; D/C, oral decitabine/cedazuridine; CI, confidence interval; CV, coefficient of variation.

The difference in maximum % LINE-1 demethylation between oral decitabine/cedazuridine and IV decitabine in cycles 1 and 2 was <1%, with a narrow CI including 0 (Table 3). Maximum % LINE-1 demethylation was numerically lower for both treatments in cycle 2 than in cycle 1; no statistical comparison was made between those values.

TABLE 3
Pharmacodynamics: Mean Maximum % LINE-1 Demethylation.

| | | | | Max. % LINE-1 | | Difference Between | |
|---|---|---|---|---|---|---|---|
| | | | Mean | Demethylation | | Oral and IV Therapy | |
| Cycle | n | Treatment | Baseline | LSM (95% CI) | | Estimate (95% CI) | |
| 1 | 62 | Oral D/C | 74.858 | 13.289 | (11.798, 14.780) | −0.730 | (−2.838, 1.378) |
| | 62 | IV decitabine | 75.523 | 14.019 | (12.528, 15.510) | | |
| 2 | 63 | Oral D/C | 73.249 | 11.151 | (9.685, 12.616) | −0.818 | (−2.890, 1.255) |
| | 63 | IV decitabine | 73.624 | 11.968 | (10.503, 13.434) | | |

D/C, decitabine/cedazuridine;
CI, confidence interval;
LINE-1, long interspersed nuclear element-1;
LSM, least squares means

Efficacy: In all, 88% of patients (117/133) were evaluable for efficacy. Reasons for not being evaluable for efficacy included absence of follow-up bone marrow results or other data required for full assessment.

Overall response rate was 62% among the total population, including those not evaluable for response (133/133). Of the 117 evaluable patients, 70% displayed a clinical response, with 25% reaching CR (Table 4). Median times to first and best responses were ˜2 months (58 days [range 7-340]) and ˜3.3 months (100 days [range 28-570]), respectively. Median durations of best response and CR were ˜1 year (371 days [95% CI 289, 439]) and ˜14 months (430 days [95% CI 355, 568]), respectively.

TABLE 4
Analysis of Best Response.

| | All Patients | | Evaluable Patients | |
|---|---|---|---|---|
| Response, n (% [95% CI]) | (n = 133) | | (n = 117) | |
| Complete response | 29 | (22 [15.1, 29.8]) | 29 | (25 [17.3, 33.6]) |
| Partial response | 0 | | 0 | |
| Marrow complete response | 43 | (32 [24.5, 41.0]) | 43 | (37 [28.0, 46.2]) |
| Marrow complete response with | 22 | (17 [10.7, 24.0]) | 22 | (19 [12.2, 27.1]) |
| hematologic improvement | | | | |
| Hematologic improvement | 10 | (8 [3.7, 13.4]) | 10 | (9 [4.2 15.2]) |
| Erythroid response | 2 | (2 [0.2, 5.3]) | 2 | (2 [0.2, 6.0]) |
| Neutrophil response | 1 | (1 [0.0, 4.1]) | 1 | (1 [0.0, 4.7]) |
| Platelet response | 7 | (5 [2.1, 10.5]) | 7 | (6 [2.4, 11.9]) |
| Total responders* | 82 | (62 [52.8, 69.9]) | 82 | (70 [60.9, 78.2]) |
| Progressive disease | 6 | (5 [1.7, 9.6]) | 6 | (5 [1.9, 10.8]) |
| No response^{†} | 29 | (22 [15.1, 29.8]) | 29 | (25 [17.3, 33.6]) |

| | | | |
|---|---|---|---|
| Not evaluable | 16 | (12 [7.0, 18.8]) | — |

*Total of all response categories.
^{†}Includes stable disease. CI, confidence interval.

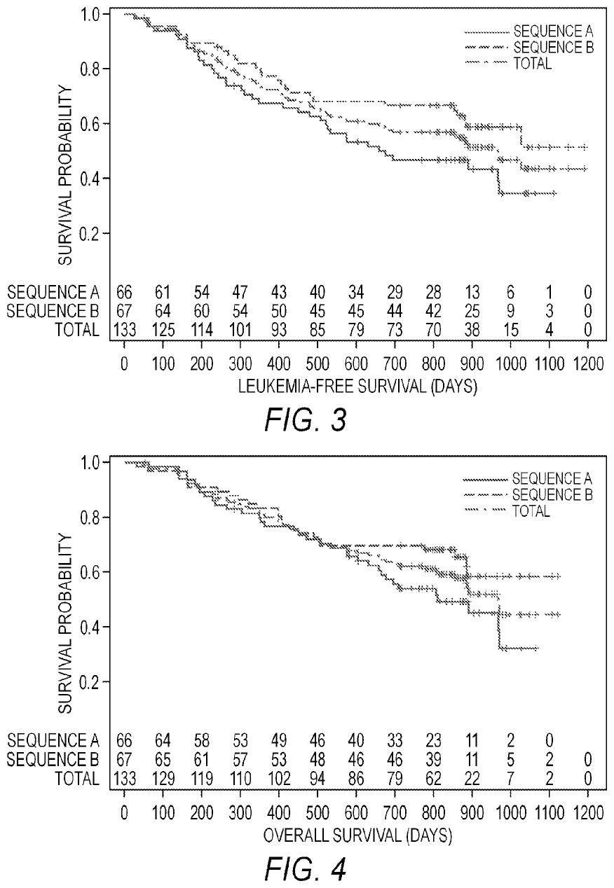

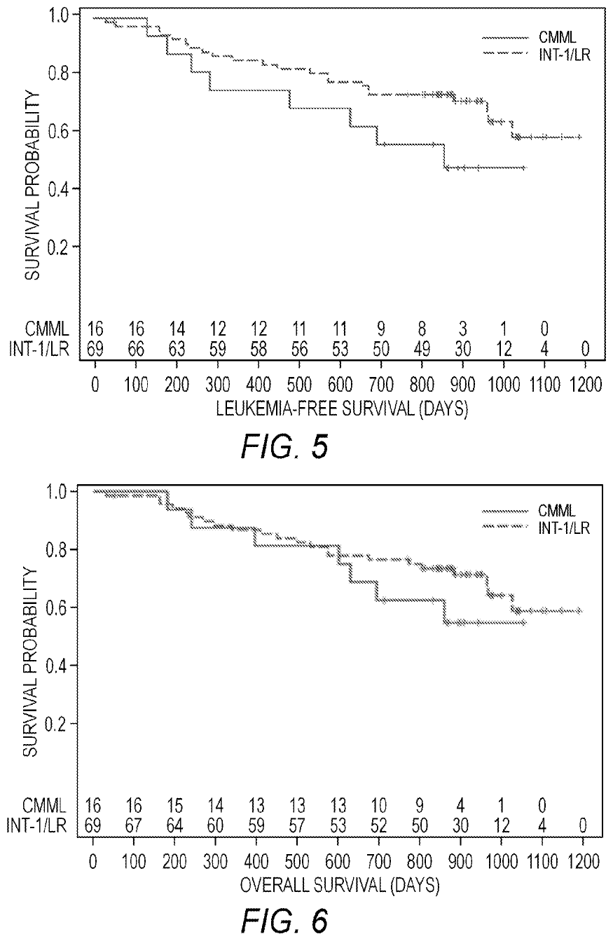

FIG. 3 is a Kaplan-Meier plot for leukemia-free survival of the subjects. FIG. 4 is a Kaplan-Meier plot for the overall survival of the subjects. FIG. 5 is a Kaplan-Meier plot for leukemia-free survival of the subjects with CMML or Int-1 or low risk MDS. FIG. 6 is a Kaplan-Meier plot for the overall survival of the subjects with CMML or Int-1 or low risk MDS. For FIGS. 3-4, Sequence A refers to subjects having oral decitabine/cedazuridine in Cycle 1 and IV decitabine in Cycle 2. Sequence B refers to subjects having IV decitabine in Cycle 1 and oral decitabine/cedazuridine in Cycle 2.

The median overall survival for all MDS subjects (overall population) is 966 days, or approximately 32 months. This is significantly higher than the overall median survival seen with treatment of MDS with azacitidine. This is also significantly higher than the overall median survival seen with treatment of MDS with decitabine alone. As such, administering oral cedazuridine and decitabine to treat MDS patients provides an unexpectedly superior improvement in median overall survival.

Among treated patients, 41% (54/133) were RBC-transfusion dependent at baseline. More than half of those 54 patients (n=28 [52%]) became RBC-transfusion independent during the study. Likewise, half of patients (6/12) who required platelet transfusions at baseline became platelet-transfusion independent. Approximately ⅓ of patients in each transfusion category were transfusion-independent for ≥112 consecutive days.

Less than half of treated patients (47%; 62/133) progressed to AML or death. Median LFS and OS were ˜29 months (889 days) and ˜32 months (966 days), respectively; 44% of patients (n=58) died during the study.

Safety: The proportions of patients with any adverse event (AE) in the first 2 treatment cycles were similar with oral and IV therapy (98% and 96%, respectively; Table 5). Most serious AEs were considered unrelated to study treatment. However, 8% of patients (11/133) had fatal treatment-emergent serious AEs during the study. Five (5/11) of these deaths were deemed related to study medication, two to oral therapy (sepsis and pneumonia) and three to IV treatment (septic shock [n=2] and pneumonia [n=1]). The rates of treatment discontinuation due to an AE during the first 2 treatment cycles was low (1 patient each) and identical in each group. No clinically meaningful increase in AEs attributed to oral treatment was observed.

TABLE 5
Most Common Treatment-Emergent Adverse Events: Cycles 1 and 2*

| Patients, n (%) | IV Decitabine (n = 132) | | Oral D/C (n = 130) | |
|---|---|---|---|---|
| ≥1 TEAE regardless of | 127 | (96) | 127 | (98) |
| relation to treatment^{†} | | | | |
| Thrombocytopenia | 52 | (39) | 63 | (48) |
| Neutropenia | 45 | (34) | 52 | (40) |
| Anemia | 47 | (36) | 49 | (38) |
| Fatigue | 23 | (17) | 26 | (20) |
| Nausea | 23 | (17) | 23 | (18) |
| Constipation | 26 | (20) | 22 | (17) |
| Diarrhea | 14 | (11) | 19 | (15) |
| Headache | 19 | (14) | 19 | (15) |
| Decreased appetite | 7 | (5) | 14 | (11) |
| Dyspnea | 13 | (10) | 15 | (12) |
| Febrile neutropenia | 9 | (7) | 18 | (14) |
| ≥1 Grade ≥3 TEAE regardless | 89 | (67) | 97 | (75) |
| of relation to treatment^{‡} | | | | |
| Neutropenia | 38 | (29) | 50 | (38) |
| Thrombocytopenia | 42 | (32) | 50 | (38) |
| Anemia | 42 | (32) | 39 | (30) |
| Leukopenia | 18 | (14) | 23 | (18) |
| Febrile neutropenia | 9 | (7) | 18 | (14) |
| Pneumonia | 7 | (5) | 7 | (5) |

*Treatment-emergent adverse events (TEAEs) were coded using Medical Dictionary for Regulatory Activities Version 22.0 and are listed in descending order of incidence in oral decitabine/cedazuridine (D/C) population.
*≥10% of oral decitabine/cedazuridine population.
^{†}≥5% of oral decitabine/cedazuridine population.

The most common treatment-emergent AEs deemed related to treatment with oral decitabine/cedazuridine were neutropenia, thrombocytopenia, anemia, leukopenia, fatigue, and nausea. Decreased appetite and nausea occurred in numerically higher proportions of patients receiving oral decitabine/cedazuridine than IV decitabine in the first 2 treatment cycles.

### Discussion

This study is the first phase 3 trial to demonstrate equivalent systemic exposure between the oral and IV formulations of a DNMTi. Systemic AUC exposures were equivalent between oral and IV dosing (geometric LSM ratio 99% [90% CI 93%, 106%; Table 2). Demethylation rates differed by <1%, demonstrating pharmacodynamic equivalence (Table 3). Findings show little difference in demethylation between sequences (i.e., whether oral or IV therapy was administered first). Effects are similar to those reported in the phase 2 study comparing oral decitabine/cedazuridine and IV decitabine therapy.¹² These findings support the conclusion that the biological effects of oral decitabine/cedazuridine and IV decitabine are similar.

The frequency of administration—once daily for the first 5 days of each 28-day treatment cycle—is identical for both formulations. Based on these findings, the US Food and Drug Administration approved oral decitabine/cedazuridine for the same indications as IV decitabine in July 2020.^{14,17}

The safety profile of oral decitabine/cedazuridine was comparable to that of IV decitabine.¹³ While treatment-emergent AEs and deaths were similar in both arms of the study, the proportion of patients reporting ≥1 treatment-related serious AE in the first 2 treatment cycles appeared to be higher among patients treated with oral therapy (9% vs 3%). One patient developed a treatment-related serious AE while receiving oral therapy in cycle 2, but an investigator deemed the AE related to IV decitabine given in cycle 1. Recalculation to account for this difference yielded serious AE rates of 8% and 4% with oral and IV therapy, respectively.

In a high proportion of patients (7/11), the treatment-related serious AE on oral therapy was febrile neutropenia. Patients who received oral therapy first (cycle 1) had a higher rate of baseline neutropenia, consistent with the cases of febrile neutropenia. Patients who received oral therapy first also had higher rates of higher risk MDS and prior decitabine treatment than those receiving IV therapy first. It is, however, unclear whether these factors affected the safety findings. Further analysis suggested that random variation may account for the higher rate of treatment-related serious AEs among patients receiving oral therapy in the first 2 cycles.

While caution should be exercised when comparing response and survival rates across trials, the overall response rate of 62% in the present study compares favorably with overall response rates reported in clinical studies of IV decitabine (32%) and the other DNMTi parenteral azacitidine (44%-60%).^{13,18-20} Likewise, the median survival of 32 months in this study exceeds previous reports of IV decitabine (14.0-19.4 months) and parenteral azacitidine (12.0-24.5 months) in clinical trials.^{13,18-21} Real-world survival is, however, lower than that observed in clinical trials. Median OS rates in two SEER-Medicare-based analyses among patients receiving DNMTi therapy were 12.3 and 13.1 months.^{22,23}

The proportion of patients in the trial who moved to transplantation reached 20%, exceeding previous transplantation rates in patients receiving DNMTi's for MDS and CMML,^{18,24} and suggesting that oral decitabine/cedazuridine may be used as a bridge to transplantation.

Oral formulations of cancer therapies are important for patients' health-related quality of life. The majority of patients receiving parenteral DNMTi therapy reported in an online survey that IV and subcutaneous treatment interfered with their social activities (72% and 81%, respectively) and daily activities (70% and 87%), and caused pain (66% and 92%).⁴ A literature review reported that in general, patients prefer oral to IV cancer therapies and value convenience, as well as the ability to receive treatment at home.²⁵ These findings may explain, at least in part, an apparent underuse of DNMTi's in patients with high-risk MDS (N=1190; high rate of not initiating [44%] and not maintaining [25%; defined as receiving <4 cycles or a ≥90-day gap between cycles] DNMTi therapy) in a real-world study using the SEER-Medicare linked data resource.²² Older age (>80 years), being unmarried, having more comorbidities, and poor performance status were associated with less likelihood of using DNMTi therapy. It is estimated that 45% of patients with MDS who initiate DNMTi therapy receive fewer than the 4 cycles of therapy recommended for each agent, despite evidence that early discontinuation is associated with relapse and poor prognosis.²³ These findings underline the need to reduce the burden of therapy for patients and improve health-related quality of life during treatment.

CC-486, an oral form of the DNMTi azacitidine, is now available as a maintenance therapy for AML after first CR after demonstrating improved OS over placebo in patients aged >55 years who received an average of one cycle of consolidation chemotherapy while in CR. CC-486 is not approved for treatment of MDS or CMML; however, it has been tested in prolonged dosing schemata (e.g., 14-21 days of a 28-day cycle in AML or MDS),^{26,27} and in a phase 3 study in lower-risk MDS.²⁸ In the latter study, it resulted in increased early death rate, despite improvement in RBC transfusion independence. While CC-486/oral azacitidine is active, it is not bioequivalent to or interchangeable with the injectable form of azacitidine used for MDS and CMML. An oral combination of cedazuridine/azacitidine in patients with MDS, CMML, or AML is in progress (ClinicalTrials.gov NCT04256317).

A strength of the present study is its crossover design in which patients served as their own control, thereby facilitating intrapatient comparison between the oral and IV formulations during the first 2 treatment cycles. All patients received oral decitabine/cedazuridine after cycle 2, and thus the safety and efficacy of the oral and IV formulations were not compared for the full duration of the trial. Given the bioequivalence of oral decitabine/cedazuridine and parenteral decitabine and a clinical profile consistent with IV decitabine, a noninferiority assessment was not deemed necessary per the regulatory authorities.

In summary, this is the first phase 3 trial to demonstrate pharmacologic equivalence between an oral and an IV formulation of a DNMTi for use in the treatment of patients with MDS or CMML. Data are in preparation from a pharmacokinetics study of oral decitabine/cedazuridine in patients with refractory anemia with excess blasts in transformation or AML with <30% blasts. The availability of an oral DNMTi reduces the treatment burden for patients, potentially increasing the initiation of and persistence with this therapy, and improving OS and patients' health-related quality of life. It also facilitates the development of all-oral combination therapy for MDS. Venetoclax, an oral B-cell lymphoma-2 protein, is under study in MDS in combination with oral decitabine/cedazuridine (ClinicalTrials.gov NCT04655755).

### REFERENCES

1. Cazzola M. Myelodysplastic syndromes. N Engl J Med 2020; 383:1358-74.
2. Greenberg P L, Stone R M, Al-Kali A, et al. NCCN Guidelines® Insights: Myelodysplastic Syndromes, Version 3.2022. J Natl Compr Canc Netw 2022; 20:106-17.
3. Cabrero M, Jabbour E, Ravandi F, et al. Discontinuation of hypomethylating agent therapy in patients with myelodysplastic syndromes or acute myelogenous leukemia in complete remission or partial response: retrospective analysis of survival after long-term follow-up. Leuk Res 2015; 39:520-4.
4. Zeidan A M, Jayade S, Schmier J, et al. Injectable hypomethylating agents for management of myelodysplastic syndromes: patients' perspectives on treatment. Clin Lymphoma Myeloma Leuk 2022; 22:e185-98.
5. Zeidan A M, Salimi T, Epstein R S. Real-world use and outcomes of hypomethylating agent therapy in higher-risk myelodysplastic syndromes: why are we not achieving the promise of clinical trials? Future Oncol 2021; 17:5163-75.
6. Griffiths E A. Oral hypomethylating agents: beyond convenience in MDS. Hematology Am Soc Hematol Educ Program 2021; 2021:439-47.
7. FDA approves new therapy for myelodysplastic syndromes (MDS) that can be taken at home. Silver Spring, M D: U.S. Food & Drug Administration, Jul. 7, 2020. (https://www.fda.gov/news-events/press-announcements/fda-approves-new-therapy-myelodysplastic-syndromes-mds-can-be-taken-home.)
8. Cinar P, Kubal T, Freifeld A, et al. Safety at the time of the COVID-19 pandemic: how to keep our oncology patients and healthcare workers safe. J Natl Compr Canc Netw 2020:1-6.
9. Mistry B, Jones M M, Kubiak P, et al. A Phase 1 study to assess the absolute bioavailability and safety of an oral solution of decitabine in subjects with myelodysplastic syndromes (MDS). Blood 2011; 118:3801.
10. Garcia-Manero G, Gore S D, Cogle C, et al. Phase 1 study of oral azacitidine in myelodysplastic syndromes, chronic myelomonocytic leukemia, and acute myeloid leukemia. J Clin Oncol 2011; 29:2521-7.
11. Savona M R, Odenike O, Amrein P C, et al. An oral fixed-dose combination of decitabine and cedazuridine in myelodysplastic syndromes: a multicentre, open-label, dose-escalation, phase 1 study. Lancet Haematol 2019; 6:e194-203.
12. Garcia-Manero G, Griffiths E A, Steensma D P, et al. Oral cedazuridine/decitabine for MDS and CMML: a phase 2 pharmacokinetic/pharmacodynamic randomized crossover study. Blood 2020; 136:674-83.
13. Steensma D P, Baer M R, Slack J L, et al. Multicenter study of decitabine administered daily for 5 days every 4 weeks to adults with myelodysplastic syndromes: the alternative dosing for outpatient treatment (ADOPT) trial. J Clin Oncol 2009; 27:3842-8.
14. Dacogen. Prescribing information. Otsuka America Pharmaceutical, Inc.; 2021.
15. Yang A S, Estecio M R, Doshi K, Kondo Y, Tajara E H, Issa J P. A simple method for estimating global DNA methylation using bisulfite PCR of repetitive DNA elements. Nucleic Acids Res 2004; 32:e38.
16. Cheson B D, Greenberg P L, Bennett J M, et al. Clinical application and proposal for modification of the International Working Group (IWG) response criteria in myelodysplasia. Blood 2006; 108:419-25.
17. Ingovi. Prescribing information. Otsuka Pharmaceutical Co., Ltd.; 2020.
18. Fenaux P, Mufti G J, Hellstrom-Lindberg E, et al. Efficacy of azacitidine compared with that of conventional care regimens in the treatment of higher-risk myelodysplastic syndromes: a randomised, open-label, phase III study. Lancet Oncol 2009; 10:223-32.
19. Hasegawa K, Wei A H, Garcia-Manero G M, et al. Clinical outcomes associated with azacitidine monotherapy for treatment-naïve patients with higher-risk myelodysplastic syndrome (H R MDS): a systematic literature review and meta-analysis. Presented at EHA 2022; Jun. 9-17, 2022; Vienna, Austria; abstr P774.
20. Silverman L R, Demakos E P, Peterson B L, et al. Randomized controlled trial of azacitidine in patients with the myelodysplastic syndrome: a study of the cancer and leukemia group B. J Clin Oncol 2002; 20:2429-40.
21. Kantarjian H, Issa J P, Rosenfeld C S, et al. Decitabine improves patient outcomes in myelodysplastic syndromes: results of a phase III randomized study. Cancer 2006; 106:1794-803.
22. Corman S, Joshi N, Wert T, Kale H, Hill K, Zeidan A M. Under-use of hypomethylating agents in patients with higher-risk myelodysplastic syndrome in the United States: a large population-based analysis. Clin Lymphoma Myeloma Leuk 2021; 21:e206-11.
23. Stein E M, Bonifacio G, Latremouille-Viau D, et al. Treatment patterns and outcomes in patients with myelodysplastic syndromes treated with hypomethylating agents: a SEER-Medicare analysis. Leuk Lymphoma 2021; 62:1411-21.
24. Sekeres M A, Schoonen W M, Kantarjian H, et al. Characteristics of U S patients with myelodysplastic syndromes: results of six cross-sectional physician surveys. J Natl Cancer Inst 2008; 100:1542-51.
25. Eek D, Krohe M, Mazar I, et al. Patient-reported preferences for oral versus intravenous administration for the treatment of cancer: a review of the literature. Patient Prefer Adherence 2016; 10:1609-21.
26. Savona M R, Kolibaba K, Conkling P, et al. Extended dosing with CC-486 (oral azacitidine) in patients with myeloid malignancies. Am J Hematol 2018; 93:1199-206.
27. Wei A H, Dohner H, Pocock C, et al. Oral azacitidine maintenance therapy for acute myeloid leukemia in first remission. N Engl J Med 2020; 383:2526-37.
28. Garcia-Manero G, Santini V, Almeida A, et al. Phase III, randomized, placebo-controlled trial of CC-486 (oral azacitidine) in patients with lower-risk myelodysplastic syndromes. J Clin Oncol 2021; 39:1426-36.

### Example 2: Prolonged Survival in Bi-Allelic TPS3-Mutated MDS Subjects Treated with Oral Decitabine/Cedazuridine

TP53 mutations (TP53mut) in MDS patients have been characterized as an independent prognostic factor for poor outcome. These patients may have similar response rates to hypomethylating agents (HMAs) but markedly diminished overall survival (OS) compared to those with wild-type (WT) TP53 status (9.4 vs. 20.7 months; See Takahashi et al., Oncotarget, 2016, 7:14172-14187). Further analyses have defined monoallelic (MA) and bi-allelic (BA)/multi-hit TP53mut populations with very different survival outcomes (8.4 vs. 30 months; See Bernard et al., Nat. Med., 2020 October; 26(10):1549-1556). Oral decitabine/cedazuridine (ASTX727) is a fixed dose combination of decitabine (35 mg) and the cytidine deaminase inhibitor cedazuridine (100 mg) with pharmacokinetic (PK) exposure equivalent to the standard intravenous (IV) decitabine regimen of 20 mg/m² daily×5 days on a 28-day cycle. The ASCERTAIN study enrolled MDS and chronic myelomonocytic leukemia (CMML) subjects and the primary endpoint demonstrating PK (AUC) equivalent of oral decitabine/cedazuridine compared with IV decitabine was met (See Garcia-Manero et al., “Pharmacokinetic Exposure Equivalence and Preliminary Efficacy and Safety from a Randomized Cross Over Phase 3 Study (ASCERTAIN study) of an Oral Hypomethylating Agent ASTX727 (cedazuridine/decitabine) Compared to IV Decitabine,” ASH 2019); median overall survival (mOS) was 31.7 months (See Savona et al., “Prolonged Survival Observed in 133 MDS Patients Treated with Oral Decitabine/Cedazuridine,” 2021 Virtual 16^{th} MDS Conference). A preliminary analysis of the mutation profile of subjects enrolled on ASCERTAIN was performed and the impact on overall and leukemia-free survival was evaluated based on the NCCN MDS panel with a focus on the TP53 mutant population. [0149] 133 subjects with MDS/CMML were enrolled to ASCERTAIN and were randomly assigned either IV decitabine for cycle 1 and oral decitabine/cedazuridine for cycle 2 or the opposite treatment sequence. All subjects continuing beyond cycle 2 received oral decitabine/cedazuridine fir all subsequence cycle until treatment discontinuation for disease progression, toxicity, patient's decision, or hematopoietic stem cell transplantation. Whole blood collected prior to treatment was used for DNA isolation and molecular abnormalities identified using next generation sequencing (NGS) hematologic malignancy panel of 179 genes including 30 genes from the NCCN MDS panel.

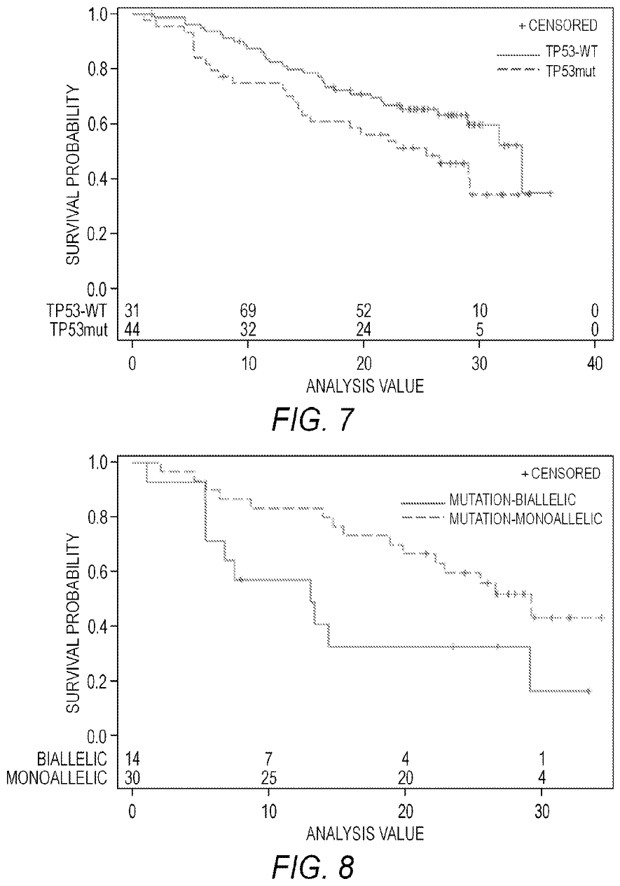

Of the 133 treated subjects, NGS analysis was available for 125 subjects. The percentage of subjects with mutations in the following genes were: TET2 (36.8%), TP53 (35.2%), ASXL1 (28%), DNMT3A (25.6%), SRSF2 MFSDI1 (17.6%), SF3B1 (15.2%), STAG2 (12.8%), EZH2 (11.2%), RUNX1 (11.2%), U2AF1 (10.4%, BCOR (10.4%), CBL (8.8%). TP53, EZH2, RUNX1, CBL, DNMT3A, SF3B1, and ASXL1 were selected for further analysis based on their reported negative impact on OS and leukemia-free survival (LFS). TP53 and CBL mutations were closely associated with a worse OS (Hazard Ratio (HR) and 95% CI: 1.70 (1.00, 2.87) and 2.54 (1.19, 5.43), respectively) and LFS (HR and 95% CI: 1.63 (0.98, 2.72) and 2.01 (0.95, 4.26), respectively) compared with WT gene status, while subjects with DNMT3A mutation showed a trending advantageous relationship with OS and LFS over WT gene status. The TP53mut population (N=44) was characterized by median age 70.5 years, 63.6% male: 36.4% female, 91% MDS: 9% CMML, IPSS categories: 20% HR, 30% Int-2, 39% Int-1, 2% LR, 9% NA, cytogenetics: 27% better-risk, 18% intermediate risk, 48% poor risk, 5% N/A, ECOG 0: 39%, 1: 61%, MA 68%, BA/multi-hit 32%. The median OS and LFS of the TP53mut population were 25.5 and 22.1 months, respectively, compared to the TP53 WT group and mOS and LFS estimates of 33.7 and 31.7 months, respectively (FIG. 7). The TP53mut population was further characterized by allelic status and found to have 14 subjects with BA mutations and 30 subjects with MA TP53 mutations without other chromosomal deletions. The respective estimated mOS and 95% CI in the BA vs. MA were 13.0 (5.3, 29.1) months vs. 29.2 (19.8, NE) months (FIG. 8).

In conclusion, the NGS mutational profile of MDS and CMML subjects in the ASCERTAIN trial included 35% with TP53mut and this group had a worse survival than those with WT TP53 apparently driven by the poor outcome of those with BA TP53mut. As a conservative estimate, treatment with oral decitabine/cedazuridine in the ASCERTAIN study resulted in an estimated survival of 13 months for BA TP53mut which compares favorably with historical results.

It will be understood by those of skill in the art that numerous and various modifications can be made without departing from the spirit of the present invention. Therefore, it should be clearly understood that the forms of the present invention are illustrative only and are not intended to limit the scope of the present invention.

All publications, patent applications, patents, patent publications, sequences identified by GenBank® database accession numbers and other references cited herein are incorporated by reference in their entireties for the teachings relevant to the sentence and/or paragraph in which the reference is presented.

The foregoing is illustrative of the present invention, and is not to be construed as limiting thereof. The invention is defined by the following claims, with equivalents of the claims to be included therein.

1. A method of treating myelodysplastic syndromes (MDS) in a subject in need thereof, comprising
administering to the subject an effective amount of cedazuridine and an effective amount of decitabine, thereby treating the MDS,
wherein administering the effective amount of cedazuridine and the effective amount of decitabine increases survival and/or leukemia-free survival of the subject to about 130% to about 400% relative to survival or leukemia-free survival, respectively, obtained by treatment with a hypomethylating agent alone; and/or provides the subject with a survival and/or leukemia-free survival of at least about 28, 30, 32, or 34 months.

2. The method of claim 1, wherein the subject is at least 75 years old.

3. (canceled)

4. The method of claim 1, wherein the effective amount of cedazuridine and the effective amount of decitabine are administered together in a combination solid oral dosage form.

5. The method of claim 1, wherein the effective amount of cedazuridine and the effective amount of decitabine are administered on days 1-5 of a 28-day cycle.

6. (canceled)

7. The method of claim 1, wherein the effective amount of cedazuridine and the effective amount of the decitabine are present in a solid oral dosage form that comprises 100 mg cedazuridine, 35 mg decitabine, and at least one pharmaceutically acceptable excipient.

8-15. (canceled)

16. The method of claim 1
wherein administering the effective amount of cedazuridine and the effective amount of decitabine increases survival and/or leukemia-free survival of the subject to about 200% to about 400% relative to survival or leukemia-free survival, respectively, obtained by providing best supportive care alone.

17-18. (canceled)

19. A method of treating lower-risk MDS in a subject in need thereof, comprising
administering to the subject an effective amount of cedazuridine and an effective amount of decitabine, thereby treating the lower-risk MDS,
wherein administering the effective amount of cedazuridine and the effective amount of decitabine increases survival and/or leukemia-free survival of the subject to about 130% to about 400% relative to survival or leukemia-free survival, respectively, obtained by treatment with a hypomethylating agent alone; and/or provides the subject with a survival and/or leukemia-free survival of at least about 28, 30, 32, or 34 months.

20-22. (canceled)

23. The method of claim 19, wherein the effective amount of cedazuridine and the effective amount of decitabine are administered on days 1-5 of a 28-day cycle.

24. (canceled)

25. The method of claim 19, wherein the effective amount of cedazuridine and the effective amount of the decitabine are present in a solid oral dosage form that comprises 100 mg cedazuridine, 35 mg decitabine, and at least one pharmaceutically acceptable excipient.

26-33. (canceled)

34. The method of claim 19
wherein administering the effective amount of cedazuridine and the effective amount of decitabine increases survival and/or leukemia-free survival of the subject to about 200% to about 400% relative to survival or leukemia-free survival, respectively, obtained by providing best supportive care alone.

35-36. (canceled)

37. A method of treating chronic myelomonocytic leukemia (CMML) in a subject in need thereof, comprising
administering to the subject an effective amount of cedazuridine and an effective amount of decitabine, thereby treating the CMML,
wherein administering the effective amount of cedazuridine and the effective amount of decitabine increases survival and/or leukemia-free survival of the subject to about 130% to about 400% relative to the survival or leukemia-free survival, respectively, obtained by treatment with a hypomethylating agent alone; and/or provides the subject with a survival and/or leukemia-free survival of at least about 28, 30, 32, or 34 months.

38-40. (canceled)

41. The method of claim 37, wherein the effective amount of cedazuridine and the effective amount of decitabine are administered on days 1-5 of a 28-day cycle.

42. (canceled)

43. The method of claim 37, wherein the effective amount of cedazuridine and the effective amount of the decitabine are present in a solid oral dosage form that comprises 100 mg cedazuridine, 35 mg decitabine, and at least one pharmaceutically acceptable excipient.

44-51. (canceled)

52. The method of claim 37
wherein administering the effective amount of cedazuridine and the effective amount of decitabine increases survival and/or leukemia-free survival of the subject to about 200% to about 375% relative to survival or leukemia-free survival, respectively, obtained by providing best supportive care alone.

53-54. (canceled)

55. A method of treating MDS or CMML in a subject having a TP53 mutation in need thereof, comprising
administering to the subject an effective amount of cedazuridine and an effective amount of decitabine, thereby treating the MDS or CMML,
wherein administering the effective amount of cedazuridine and the effective amount of decitabine increases survival and/or leukemia-free survival of the subject to about 130% to about 400% relative to survival or leukemia-free survival, respectively, obtained by treatment with a hypomethylating agent alone; and/or provides the subject with a survival and/or leukemia-free survival of at least about 20, 22, 24, 26, 28, 30, 32, 34, or 36 months for a subject with a mono-allelic TP53 mutation or at least about 10, 12, 14, 16, 18, or 20 months for a subject with a bi-allelic TP53 mutation.

56-58. (canceled)

59. The method of claim 55, wherein the effective amount of cedazuridine and the effective amount of decitabine are administered on days 1-5 of a 28-day cycle.

60. (canceled)

61. The method of claim 55, wherein the effective amount of cedazuridine and the effective amount of the decitabine are present in a solid oral dosage form that comprises 100 mg cedazuridine, 35 mg decitabine, and at least one pharmaceutically acceptable excipient.

62-69. (canceled)

70. The method of claim 55
wherein administering the effective amount of cedazuridine and the effective amount of decitabine increases survival and/or leukemia-free survival of the subject to about 200% to about 400% relative to survival or leukemia-free survival, respectively, obtained by providing best supportive care alone.

71-87. (canceled)

88. A kit comprising one or more doses (e.g., 1, 2, 3, 4, or 5) of an effective amount of cedazuridine (e.g., 100 mg) and one or more doses (e.g., 1, 2, 3, 4, or 5) of an effective amount of decitabine (e.g., 35 mg) for the use in the method of claim 1.

89. A combination comprising one or more doses (e.g., 1, 2, 3, 4, or 5) of an effective amount of cedazuridine (e.g., 100 mg) and one or more doses (e.g., 1, 2, 3, 4, or 5) of an effective amount of decitabine (e.g., 35 mg) for the use in the method of claim 1.
